(19) United States
(12) Patent Application Publication
(10) Pub. No.: US 2021/0032589 A1
(43) Pub. Date: Feb. 4, 2021

(54) METHODS AND DEVICES FOR THE ISOLATION OF SUBCELLULAR COMPONENTS

(71) Applicant: Flagship Pioneering Innovations V, Inc., Cambridge, MA (US)

(72) Inventors: Michael Mee, Boston, MA (US); Adam Rago, Somerville, MA (US); Geoffrey von Maltzahn, Somerville, MA (US); John Miles Milwid, Winchester, MA (US); Jacob Rosenblum Rubens, Cambridge, MA (US); Michael J. Cima, Winchester, MA (US)

(73) Assignee: Flagship Pioneering Innovations V, Inc., Cambridge, MA (US)

(21) Appl. No.: 16/968,246

(22) PCT Filed: Feb. 8, 2019
(86) PCT No.: PCT/US2019/017264

§ 371 Date: Aug. 7, 2020

Related U.S. Application Data
(60) Provisional application No. 62/629,255, filed on Feb. 12, 2018.

(51) Int. Cl. C12M 1/00; C12N 1/06; C12M 1/42; C12M 1/26

(52) U.S. Cl. CPC C12M 47/06; C12N 1/066; C12M 35/04; C12N 2521/00; C12M 33/10; C12N 2527/00; C12M 35/02

(57) ABSTRACT

One aspect of the invention provides an apparatus for isolating one or more subcellular components including a cell disruption reservoir that generates at least one of a phase change, a thermal change, a physical contact force, an ultrasonic frequency, an osmotic change, a pressure change, a photothermal pulse, a magnetic field, an electromagnetic field, an electric field, and an electrical pulse through the reservoir and a separation instrument configured to specifically isolate the subcellular components based on one or more parameters selected from at least one of density, charge/pH, dielectric polarization, magnetic attraction, spectral dispersion, spectral refraction, spectral diffraction, hydrophobicity, hydrophilicity, structure (presence or absence of a structural feature), function (migration), affinity or binding, and pressure.

### CROSS-REFERENCE TO RELATED APPLICATIONS

This application claims the benefit of priority to U.S. Provisional Patent Application Ser. No. 62/629,255, filed Feb. 12, 2018. The entire content of this application is hereby incorporated by reference herein.

### BACKGROUND OF THE INVENTION

Cells include a variety of subcellular components, also known as organelles, that have a specific function.

There remains a need in the art for methods and devices capable of efficiently sorting and isolating subcellular components.

### SUMMARY OF THE INVENTION

One aspect of the invention provides an apparatus for isolating one or more subcellular components including a cell disruption reservoir that generates at least one of a phase change, a thermal change, a physical contact force, an ultrasonic frequency, an osmotic change, a pressure change, a photothermal pulse, a magnetic field, an electromagnetic field, an electric field, and an electrical pulse through the reservoir and a separation instrument configured to specifically isolate the subcellular components based on one or more parameters selected from at least one of density, charge/pH, dielectric polarization, magnetic attraction, spectral dispersion, spectral refraction, spectral diffraction, hydrophobicity, hydrophilicity, structure (presence or absence of a structural feature), function (migration), affinity or binding, and pressure.

This aspect of the invention can include a variety of embodiments. The cell disruption reservoir can generate a photothermal pulse. The cell disruption reservoir can generate a pressure change. The cell disruption reservoir can include an inlet and an outlet for fluidic movement that generates the osmotic change.

The separation instrument can include a centrifuge.

The subcellular components can include organelles.

Another aspect of the invention provides an apparatus for isolating one or more subcellular components including a reservoir comprising an inlet and an outlet for fluidic movement into and out of the reservoir, a pump to regulate a fluid flow through the reservoir and a separation instrument configured to specifically isolate the subcellular components based on one or more parameters selected from at least one of density, charg/pH, magnetic attraction, spectral dispersion, spectral refraction, spectral diffraction, hydrophobicity, hydrophilicity, structure (presence or absence of a structural feature), and function (migration).

This aspect of the invention can include a variety of embodiments. The reservoir can further include a channel having a diameter 20-90% of an input component to physically contact the input component as the pump fluidically forces the input component through the channel. The reservoir further can further include a cell disruption homogenizing member to physically contact an input component with a physical contact force. The separation instrument can include a centrifuge. The subcellular components can be organelles.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIGS. 1A-1D depict schematics of embodiments of an apparatus for isolating one or more subcellular components from a cell according to embodiments of the invention.

FIGS. 2A and 2B depict a tissue homogenizer cell disruption device according to an embodiment of the invention utilizing a rotating pestle.

FIG. 2C depicts a tissue homogenizer cell disruption device according to an embodiment of the invention utilizing a ball bearing.

FIG. 3 depicts a microfluidic cell disruption device according to an embodiment of the invention.

FIG. 4 depicts a sonication cell disruption device according to an embodiment of the invention.

FIGS. 5A-5D depict a gas cavitation cell disruption device according to an embodiment of the invention.

FIGS. 6A-6D depict a temperature controlled cell disruption device according to an embodiment of the invention.

FIG. 7 depicts a photo disruption device according to an embodiment of the invention.

FIGS. 8A-8C depict a projectile force cell disruption device according to an embodiment of the invention.

FIGS. 9A and 9B depict a chemical disruption device according to an embodiment of the invention.

FIG. 10 depicts an imaging and detection apparatus for subcellular component separation, comprising a camera, a microscope and a computer according to an embodiment of the invention.

FIG. 11 depicts a filtration device for the isolation of subcellular components according to an embodiment of the invention.

FIGS. 12A-12G depict density gradient subcellular component separation apparatuses according to embodiments of the invention. FIGS. 12A-12C depict density gradient apparatuses utilizing two or more fluid phases to separate subcellular components. FIGS. 12D-12G depict density gradient apparatuses which separate subcellular components by density and specific gravity by sequential centrifugation and pelleting.

FIGS. 13A and 13B depict magnetic separation devices for the separation of subcellular components, according to embodiments of the invention. FIG. 13A depicts the magnetic separation device while the magnetic field is active and FIG. 13B depicts the magnetic separation device while the magnetic field is inactive.

FIGS. 14A and 14B depict high-throughput size retention devices for the separation of subcellular components according to embodiments of the invention.

### DETAILED DESCRIPTION OF THE INVENTION

Embodiments of the invention provide a variety of devices and methods for isolation of subcellular components (also known as “organelles”) from cells.

### Isolation of Subcellular Components

Embodiments of the invention are particularly useful for the isolation of subcellular components, such as mitochondria, from the bulk materials of a cell. Such isolated subcellular components can be then administered to a subject (optionally after further processing). The invention can be adapted by a person of ordinary skill in the art for the isolation of any organelle of a typical prokaryotic or eukaryotic cell. For example, the invention can be adapted and configured for the isolation of mitochondria, chloroplasts or cell nuclei.

Embodiments of the invention are particularly useful for the preparation of subcellular components such as mitochondria. Compositions including isolated subcellular components such as mitochondria are described in U.S. Patent Application Publication No. 2017/0151287.

Embodiments of the invention can be utilized, in whole or in part, to prepare chondrisomes, chondrisome preparations, fusogens, fusosomes, and/or fusosome compositions, as further described in the Appendix.

### Apparatus for Isolating Subcellular Components

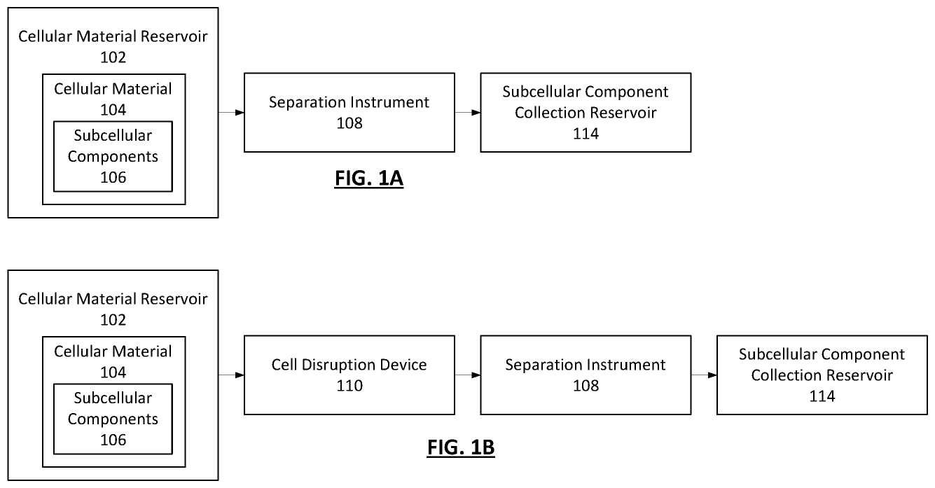

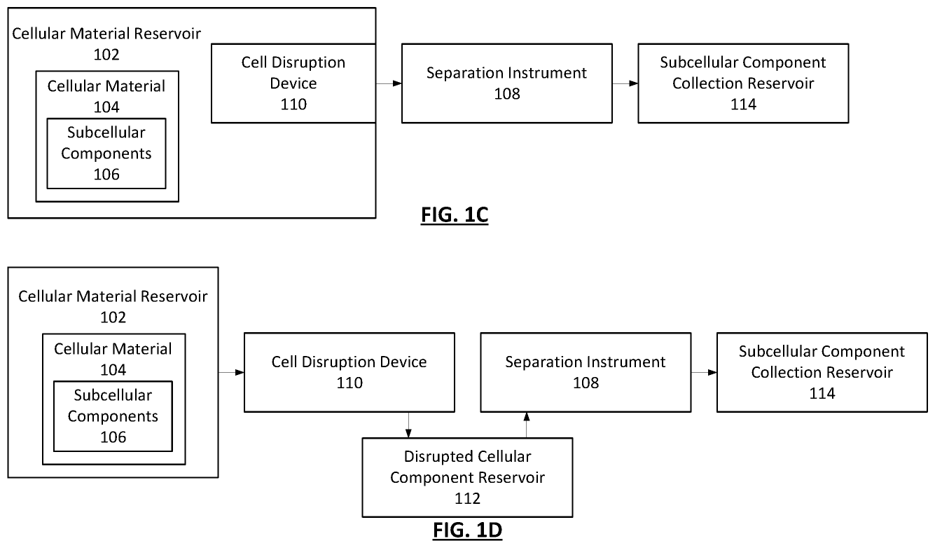

Referring now to FIGS. 1A-1D, one embodiment of the invention provides an apparatus 100 for isolating one or more subcellular components from a cell. The apparatus includes a cellular material reservoir 102 for holding cellular material 104 including the subcellular components 106 and a separation instrument 108 configured to specifically isolate the subcellular components 106 based on one or more parameters.

The apparatus 100 can further include a cell disruption device 110. In some embodiments, the cellular material reservoir 102 can include the cell disruption device 110. In some other embodiments, the cellular material reservoir 102 is in fluidic communication with the cell disruption device 110, which is, in turn, in fluidic communication with the separation instrument 108. The apparatus 100 can further include a disrupted cellular component reservoir 112 in fluidic communication with the cell disruption device 110 and the separation instrument 108.

The apparatus 100 can further include a subcellular component collection reservoir 114 in fluidic communication with the separation instrument 108 for collecting the isolated subcellular components 106. The apparatus 100 can also include one or more pumps, which can aid in moving fluids from one component to the other. Additionally, the apparatus 100 can include an automated liquid handling system adapted and configured for transferring fluids from one component of the apparatus 100 to another. In certain embodiments, this automated liquid handling system can be a 3-axis robotic system fitted with one or more syringes or pipettes capable of transferring known volumes of cellular material 104 from one component to another.

In some embodiments, the cellular material 104 can include intact cells 116 that require disruption by the cell disruption device 110 in order to release the subcellular components 106. In other embodiments, the cellular material 104 comprises already-lysed cells or free-floating homogenized subcellular components 106.

Referring again to FIGS. 1A and 1B, in certain embodiments, the apparatus includes a cellular material reservoir 102 in fluidic communication with a separation instrument 108. The apparatus can also include a cellular material reservoir 102 in fluidic communication with a cell disruption device 110, which is in turn in fluidic communication with a separation instrument. One or more pumps adapted and configured to move cellular material 104 can be employed to move materials from the cellular material reservoir 102 to the cell disruption device 110, from the cell disruption device 110 to the separation instrument 108, and from the separation instrument 108 to the subcellular component collection reservoir 114. Alternatively, the components can be fluidically isolated from one another and the apparatus can comprise one or more robotic devices fitted with a means to transfer cellular material 104 from one component to the other. The robotic devices can be fitted with syringes or pipettes adapted and configured to draw cellular material 104 and transport it from one component to another.

The apparatus 100 can further comprise a control unit 116 programmed to control operation of one or more components of the invention selected from one or more pumps adapted and configured to move cellular material 104, one or more robotic devices fitted with a means of transferring cellular material, the cell disruption device 110, and the separation instrument 108.

Once subcellular components 106 have been isolated within the subcellular component collection reservoir 114, they can be concentrated further, for example, by centrifuging. In one embodiment, the isolated subcellular components 106 can be further isolated by centrifuging at 9,000 g for 10 minutes at about 4° C., although the centrifuging procedure can be modified in order to obtain the optimal desired concentration.

The apparatus 100 (and other devices described herein) can be a device adapted and configured for medical use. For example, the apparatus 100 as a whole and/or all components that come in contact with cellular material 104 or subcellular components 106 can be sterile or sterilizable, in order to avoid contamination of the cellular material 104 or subcellular components 106. The apparatus 100 can also include disposable materials which can be replaced after use in order to avoid cross-contamination between different cellular material 104 or subcellular components 106. The disposable materials can be commercially available components such as disposable vials, disposable linings, disposable reservoirs and the like. The components can also be made up of materials that comply with various medical device regulations and best practices, e.g., components that do not leach or degrade into the cellular material 104 or subcellular component 106 samples.

### Cell Disruption Devices

Cell disruption devices 110 according to embodiments of the invention can be one or more of any of a number of devices known in the art which are adapted and configured to disrupt a cell in such a way that the components of the cell are released from the cellular membrane or cellular wall. Cell disruption devices 110 can disrupt the homeostasis of a cell by lysing the cell. Certain cell disruption devices 110 also homogenize the resulting cellular contents.

In certain embodiments, the cell disruption device 110 operates through one or more methods selected from the group consisting of physical cell disruption, cryogenic disruption, heat disruption, pressure disruption, chemical disruption, sonic disruption and photo disruption. The cell disruption device can generate at least one of a phase change, a thermal change, a physical contact force (e.g., shear contact force), an ultrasonic frequency, an osmotic change, a pressure change, a photothermal pulse, a magnetic field, an electromagnetic field, an electric field, and an electrical pulse.

The apparatus 100 can include two or more cell disruption devices 110 operating in sequence. In certain embodiments, the use of two or more cell disruption devices 110 in sequence can yield greater cell disruption, increasing the yield of freed subcellular components 106 and decreasing the amount of intact cells. The two or more cell disruption devices 110 operating in sequence can be one or more of the cell disruption devices 100 described herein or any equivalent devices known in the art.

The apparatus 100 can include two or more cell disruption devices 110 operating in parallel. In certain embodiments, the use of two or more cell disruption devices 110 in parallel can increase throughput of the apparatus 100. In a preferred embodiment, the apparatus can include two or more cell disruption devices 110 that are identical or substantially identical operating in parallel, feeding into one or more separation instruments 108.

In certain embodiments, the cell disruption device 110 can make use of membrane disrupting compounds in addition to the described components. For example, the cell disruption device 110 can include an enzyme solution. The enzyme solution can comprise any enzymes known in the art to aid in the disruption of cells. Cell disrupting enzymes can include collagenases, achromopeptidase, labiase, lysostaphin, lysozyme, mutanolysin, lyticase, cellulose, pecitinase, pectolyase, tetanolysin, hemolysin, stretolysin, trypsin, subtilisin, proteinase k, papain, and the like. The cellular material 104 can also or alternatively be mixed with a solution comprising one or more membrane solubilizers. Membrane solubilizers can include any membrane lysing buffer or solution known in the art, including, for example Tris-HCL solutions, EDTA solutions, TRITON™ X-100 detergent solutions, SDS (sodium dodecyl sulfate) solutions, CHAPS (3-[(3-cholamidopropyl)dimethylammonio]-1-propanesulfonate) solutions, ethyl trimethyl ammonium bromide solutions, and the like.

### Tissue Homogenizer

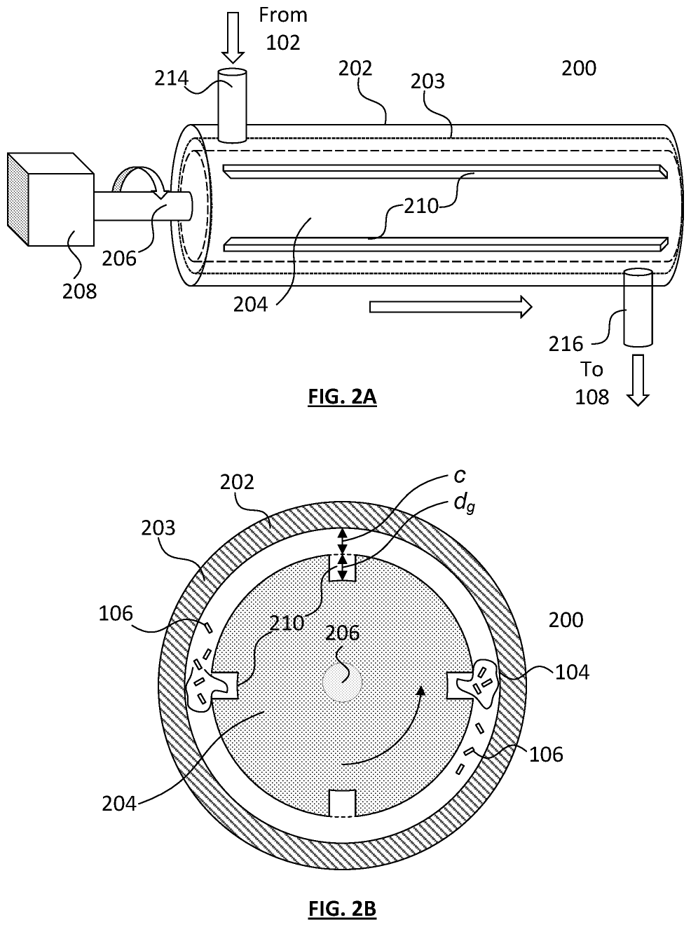

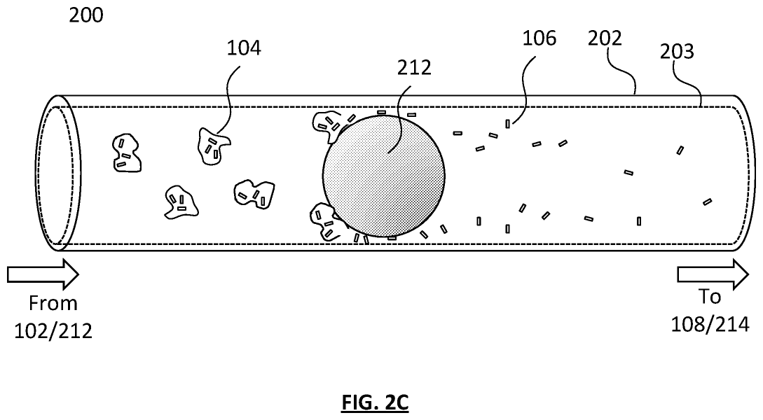

Referring now to FIGS. 2A and 2B, one embodiment of the cell disruption device 110 is a tissue homogenizer 200. The tissue homogenizer 200 can include a tubular vessel 202 having an inner wall 203. The tubular vessel 202 can receive a pestle 204 mounted to a shaft 206. The shaft 206 can be mounted to a motor 208. In certain embodiments, the pestle 204 can include one or more grooves 210 on the outer surface. The cellular material 104 can be added to the tissue homogenizer 200 in the tubular vessel 202. The pestle 204 can then be rotated at a rate sufficient to result in the breakdown of the cellular connective tissue, proteins and cell membranes, resulting in cell disruption and the release of the subcellular components 106. The motion of the pestle 204 within the tubular vessel 202 can homogenize the tissue through sheer force.

The tubular vessel 202 and the pestle 204 can be substantially any shape which allows for the rapid rotation of the pestle 204. In certain embodiments, the tubular vessel 202 and the pestle 204 are substantially cylindrically shaped or conically shaped. The pestle 204 can be oriented within the tubular vessel 202 such that any point on the grooved outer surface of the pestle is approximately the same clearance from the inner wall 203 of the tubular vessel. In certain embodiments, the distance between the outer surface of the pestle is about 5 μm to about 100 μm from the inner wall 203 of the tubular vessel, or any distance in between. In certain embodiments, the grooves 210 on the outer surface of the pestle 204 have a depth of about 1 mm to about 5 mm, or any distance in between.

In certain embodiments, the tubular vessel 202 includes a material selected from the group consisting of glass, metal, plastic and polymeric materials. The tubular vessel 202 can be made up of a material capable of withstanding a wide range of temperatures, ranging from cryogenic temperatures to at least about 100° C., and any temperature in between.

In other embodiments, the pestle 204 includes a material selected from the group consisting of polytetrafluoroethylene, metal, plastics, glass and other polymeric materials. The pestle 204 can also be made up of a material capable of withstanding a wide range of temperatures, ranging from cryogenic temperatures to at least about 100° C., and any temperature in between.

In order to lyse the cells and release the subcellular components 106, the pestle 204 can be rotated at a speed of about 100 revolutions per minute (RPM), 200 RPM, 300 RPM, 500 RPM, 750 RPM, 1000 RPM, 2000 RPM or any rotational speed in between. In certain embodiments, the rotational speed can be gradually increased or decreased.

In an alternative embodiment, referring now to FIG. 2C, the cell disruption device 110 can be a tissue homogenizer 200 including a tubular vessel 202 having an inner wall 203 which can receive a ball bearing 212. The ball bearing 212 can be substantially spherical in shape and have a diameter such that the distance between the outer surface of the ball bearing 212 is about 5 μm to about 100 μm from the inner wall 203 of the tubular vessel, or any distance in between.

The tissue homogenizer 200 having a ball bearing 212 can disrupt cellular material 104 by having the cellular material 104 flow through the tubular vessel 202 and forced past the ball bearing 212 under pressure, such that large cells are squeezed and lysed, releasing the subcellular components 106. In certain embodiments, the clearance between the ball bearing 212 and the inner wall 203 is sufficient to allow subcellular components 106 to pass but not whole cells. The ball bearing 212 can be made of metal, plastics, glass or any other suitable material hard enough to cause cellular disruption. In one embodiment, the ball bearing 212 can be made of tungsten carbide or another hard metallic alloy.

In certain embodiments, the tissue homogenizer 200 is in fluidic communication with the cellular material reservoir 102 and the separation instrument 108 such that cellular material 104 comprising the subcellular components 106 from the cellular material reservoir 102 can flow into the tissue homogenizer 200 and homogenized subcellular components 106 can flow from the tissue homogenizer 200 into the separation instrument 108. The tissue homogenizer 200 can be in fluidic communication with the cellular material reservoir 102 through an inlet 214 and with the separation instrument 108 through an outlet 216. The inlet 214 and the outlet 216 can comprise a valve adapted and configured to regulate the flow of cellular material 104 into and out of the tissue homogenizer 200.

In one potential embodiment, the tissue homogenizer 200 can further comprise heating and/or cooling elements adapted and configured to regulate the temperature within the tubular vessel 202.

In certain embodiments, the tissue homogenizer 200 can be controlled by the controlling unit 116. The controlling unit 116 can regulate flow of cellular material 104 through the inlet 214 and the outlet 216 and the rate of rotation of the pestle 204. The controlling unit 116 can also regulate the temperature within the tubular vessel 202 by controlling a heating element, a cooling element or both contained within the tissue homogenizer 200.

### Microfluidic Cell Disruptor

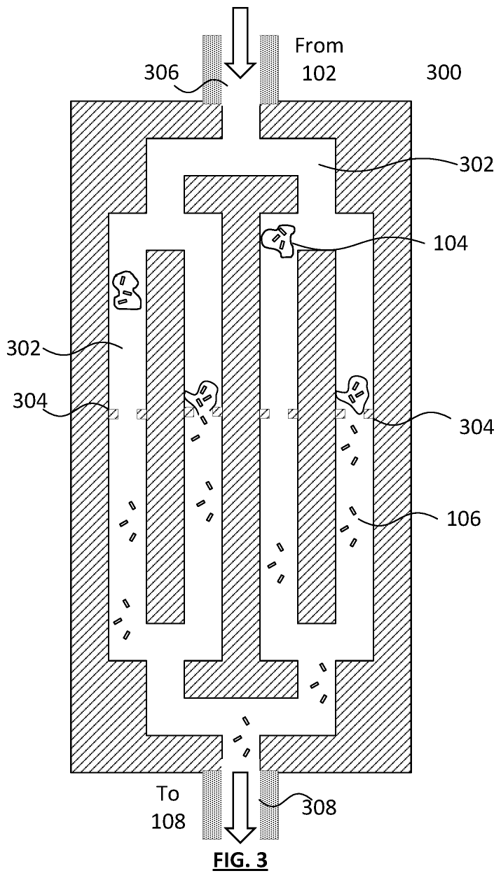

Referring now to FIG. 3, one embodiment of the cell disruption device 110 is a microfluidic cell disruptor 300. The microfluidic cell disruptor 300 can include a series of microfluidic channels 302 with a small diameter, such that cells are constricted when pumped through the channels, resulting in temporary or permanent loss of cell membrane integrity due to pressure and shear stress.

A microfluidic system 300 according to an embodiment of the invention can include microfluidic channels 302 including one or more constrictions 304. In certain embodiments, these microfluidics channels 302 can be channels etched into a solid material such as a silicon chip and sealed with a layer of a glass. The constrictions can have a diameter smaller than about 50% of the diameter of the cells 116 within the cellular material 104 that is being disrupted and larger than the diameter of the desired subcellular components 106. In certain embodiments, the constrictions can have a width of about 4-8 μm and a depth of about 10-50 μm.

The microfluidic system can include a multichannel design wherein the system comprises two or more interconnected channels 302 running in parallel such that flow through the microfluidic system 300 is not hampered by a clog or defect in any single channel.

The microfluidic channels 302 can be in fluidic communication with the cellular material reservoir 102 through an inlet 306 that joins the microfluidic system 300 with the cellular material reservoir 102 and in fluidic communication with the separation instrument 108 through an outlet 308 that joins the microfluidic system 300 with the separation instrument 108. A mixture of cellular material 104 including the subcellular components 106 contained within the cellular material reservoir 102 can be pumped through the inlet 306, through the channels of the microfluidic system, whereby the cellular material 104 is disrupted, through an outlet 308 and into the separation instrument 108. In certain embodiments, the inlet 306 and the outlet 308 can include a valve adapted and configured to regulate the flow of cellular material 104 into and out of the microfluidic cell disruptor 300.

In certain embodiments, the throughput rate through the microfluidic system 300 can be about 100 cells/s, about 500 cells/s, about 1,000 cells/s, about 5,000 cells/s, about 10,000 cells/s, about 20,000 cells/s, about 100,000 cells/s, about 1,000,000 cells/s, about 10,000,000 cells/s or any values in between.

In certain embodiments, the microfluidic cell disruptor 300 can be controlled by the controlling unit 116. The controlling unit 116 can regulate flow of cellular material 104 through the inlet 306 and through the outlet 308.

### Sonicator

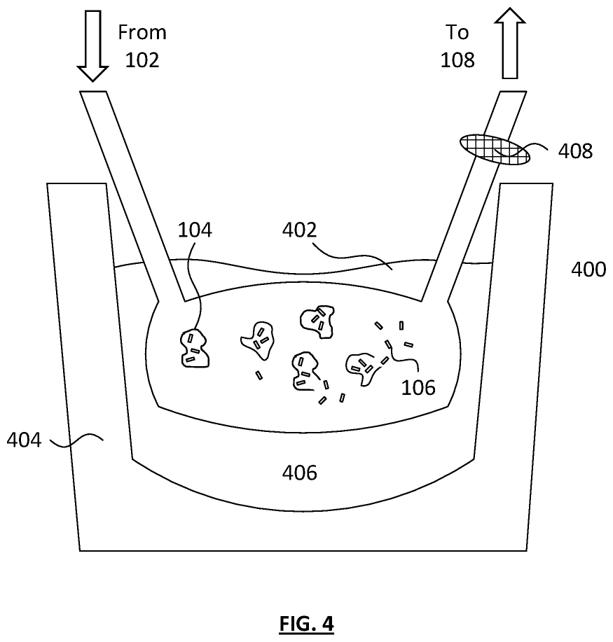

Referring now to FIG. 4, another embodiment of the cell disruption device 110 can be a sonicator 400 that can disrupt cells using energy from ultrasound waves.

In one embodiment, cellular material 104 can be placed in a sonication reservoir 402. Any air within the sonication reservoir 402 can be removed and the reservoir can be submerged in a sonication device 404 including a liquid (e.g., water) bath 406. The cellular material 104 can then be sonicated at a frequency sufficient to disrupt the cells within the cellular material 104, releasing the subcellular components 106. The cellular material 104 can then be pumped from the sonication reservoir 402, through one or more filters 408. The one or more filters 408 can be mesh filters wherein each filter has a mesh size independently selected from about 20 μm to about 500 μm and any size in between.

In certain embodiments, the cellular material reservoir 102 can be in fluidic communication with the sonication reservoir 402 such that cellular material 104 can be pumped from the cellular material reservoir 102 to the sonication reservoir 402. The sonication reservoir 402 can also be in fluidic communication with the separation instrument 108, wherein the cellular material 104 can be pumped from the sonication reservoir 402, through one or more filters and into the separation instrument 108.

The sonication device 404 can include a bath 406 with a controlled temperature. In certain embodiments, the bath 406 can be held at a temperature from about 30° C. to about 40° C. or any temperature in between, most preferably at 37° C. The sonication device 404 can also be operated at a range of sonication frequencies and powers and for different periods of time in order to sufficiently disrupt the cells. In certain embodiments, the sonication device 404 can be operated at 43 kHz at a power of about 0.9 watt/cm², although the frequency and power can be modified by a person of ordinary skill in the art in order to optimize cell disruption. The cellular material 104 can be sonicated for a period of time from about 10 minutes to about 1 hour, preferably about 20 minutes.

In certain embodiments, the cellular material 104 can be mixed with a solution comprising one or more enzymes prior to sonication. The enzyme solution can comprise any enzymes known in the art to aid in the disruption of cells. Cell disrupting enzymes can include collagenases, achromopeptidase, labiase, lysostaphin, lysozyme, mutanolysin, lyticase, cellulose, pecitinase, pectolyase, tetanolysin, hemolysin, stretolysin, trypsin, subtilisin, proteinase k and papain. The cellular material 104 can also or alternatively be mixed with a solution comprising one or more membrane solubilizers. Membrane solubilizers can include any membrane lysing buffer or solution known in the art, including, for example Tris-HCL solutions, EDTA solutions TRITON™ X-100 detergent solutions, SDS (sodium dodecyl sulfate) solutions, CHAPS (3-[(3-cholamidopropyl)dimethylammonio]-1-propanesulfonate) solutions, ethyl trimethyl ammonium bromide solutions and like.

In certain embodiments, the sonicator 400 can be controlled by the controlling unit 116. The controlling unit 116 can regulate flow of cellular material 104 into and out of the sonication reservoir 402, the temperature of the bath 406, and the power and frequency of the sonication device 404.

### Gas Cavitation Device

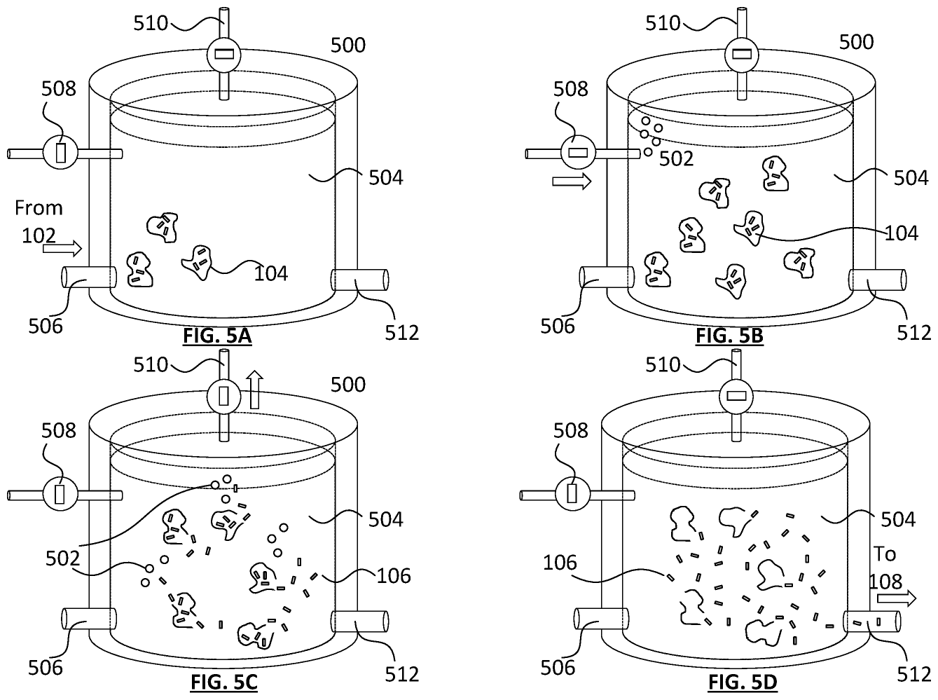

Referring now to FIGS. 5A-5D, another embodiment of the cell disruption device 110 can be a gas cavitation device 500 for disruption of cells or tissue using gas cavitation based on differential gas pressure. The gas cavitation device 500 dissolves a gas 502 within cells under high pressure within a pressure chamber 504, then rapidly releases said pressure. This causes the gas 502 to come out of solution (nucleate). Gas bubbles increase in size, stretching and ultimately disrupting cell membranes. In certain embodiments, the dissolved gas 502 is an inert gas which is soluble in aqueous solutions, such as nitrogen gas.

In one embodiment of a gas cavitation device 500, cellular material 104 dissolved in a solution can be added to a pressure chamber 504, potentially through a sample inlet 506. The pressure chamber 504 is then sealed and oxygen-free nitrogen gas is added to the chamber through a gas inlet 508, increasing the pressure of the chamber and dissolving nitrogen in the solution. Once the pressure reaches a sufficient level, the pressure in the chamber is released through a gas outlet 510 allowing the pressure to rapidly decrease back to atmospheric pressure. A sample outlet 512 on the pressure chamber can be opened to allow for the lysed cells to be collected. In certain embodiments, the cellular material reservoir 102 can be in fluidic communication with the pressure chamber such that cellular material 104 can be pumped from the cellular material reservoir 102 to the pressure chamber through the sample inlet 506. The pressure chamber 504 can also be in fluidic communication with the separation instrument 108, wherein the cellular material 104 can be pumped from the pressure chamber 504, out of the sample outlet 512 and into the separation instrument 108.

The pressure chamber 504 can be substantially cylindrical in shape and can comprise a pressure cap with a rubber gasket seal. The pressure chamber can comprise a gas inlet valve 508 configured and adapted for the addition of nitrogen gas to the pressure chamber. The pressure chamber can further comprise a gas outlet release valve 510, which can be opened to release accumulated pressure after pressurization. The pressure chamber 504 can also further comprise a pressure gauge for measuring and recording the internal pressure of the pressure chamber 504.

In certain embodiments, the pressure chamber 504 can be pressurized to a pressure of about 400 psi, about 450 psi, about 600 psi, about 750 psi, about 1,000 psi, about 2,000 psi, about 10,000, about 35,000 psi, about 50,000 psi or any pressure in between, before release in order to lyse the cells. The pressure chamber 504 can be pressurized to the above temperature for about 1 second, about 5 seconds, about 30 seconds, about 60 seconds, or any amount of time in between or any reasonable amount of time as determined by a person of ordinary skill in the art. The pressure chamber 504 can be cycled from high pressure to low pressure multiple times in order to sufficiently disrupt the cellular material 104. For example, the gas cavitation device 500 can be cycled from high pressure to low pressure for 2 cycles, 5 cycles, 10 cycles, 20 cycles, 50 cycles, 100 cycles or any number in between. The pressure can also be altered from one cycle to the next. Pressurization procedures can be easily optimized for maximum subcellular component 106 release by a person of ordinary skill in the art.

In certain embodiments, the gas cavitation device 500 can be controlled by the control unit 116. The control unit 116 can regulate flow of cellular material 104 through the sample inlet 506 and the sample outlet 512, and the rate of flow of gas 502 through the gas inlet 508 and gas outlet 510.

### Temperature-Controlled Devices

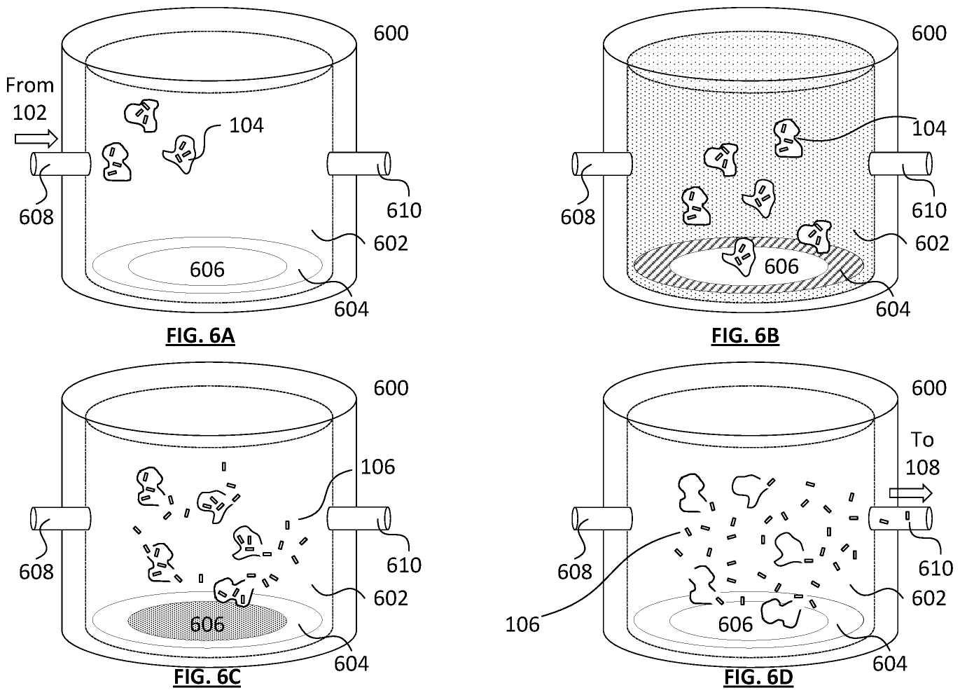

Referring now to FIGS. 6A-6D, another embodiment of the cell disruption device 110 can be a temperature controlling device 600 used to sequentially freeze and thaw cells or tissue to disrupt cellular integrity.

In one embodiment, the temperature controlling device 600 can comprise a temperature-regulated chamber 602 comprising a cooling mechanism 604 capable of lowering the temperature within the chamber 602 to temperatures below the 0° C. Exemplary cooling mechanisms 604 include thermoelectric (Peltier) coolers, adiabatic cooling devices, fluid-cooled units that communicate with an external heat exchanger, and cryogenic devices that utilize cooled gases such as nitrogen or carbon dioxide to produce the desired low temperatures. In certain embodiments, the temperature controlling device 600 is configured to lower the temperature within the temperature-regulated chamber 602 to a temperature below −10° C., below −20° C., below −30° C., below −40° C., below −50° C., and any temperatures in between.

Additionally, the temperature-regulated chamber 602 can include a warming mechanism 606 capable of raising the temperature within the temperature-regulated chamber 602 to a temperature above 0° C. Exemplary warming mechanisms include coherent light sources, incoherent light sources, heated fluid sources, resistive (Ohmic) heaters, microwave generators (e.g., producing frequencies between about 915 MHz and about 2.45 GHz), and ultrasound generators (e.g., producing frequencies between about 300 KHZ and about 3 GHz). In certain embodiments, the temperature controlling device 600 can be configured to raise the temperature within the temperature-regulated chamber 602 to a temperature above 10° C., above 20° C., above 30° C., above 40° C., above 50° C., and any temperatures in between. In one example, the warming mechanism 606 can raise the temperature of the temperature-regulated chamber 602 to 37° C.

In certain embodiments, the cellular material reservoir 102 can be in fluidic communication with the temperature controlling device 600 such that cellular material 104 can be pumped from the cellular material reservoir 102 to the temperature-regulated chamber 602. The temperature controlling device 600 can also be in fluidic communication with the separation instrument 108, wherein the subcellular components 106 can be pumped from the temperature-regulated chamber 602 into the separation instrument 108.

In an exemplary procedure, cellular material 104 can be pumped from the cellular material reservoir 102, into the temperature-regulated chamber 602 through a sample inlet 608. The cooling mechanism 604 can then cool the temperature-regulated chamber 602 to about −20° C. over a first period of time, causing the cells within the cellular material 104 to swell due to the formation of water ice crystals, ultimately lysing the cells. The temperature-regulated chamber 602 can then be warmed to a temperature of about 37° C. by the warming mechanism 606 over a second period of time, causing the cellular material 104 to thaw and contract. This cooling/heating process can be repeated one or more additional times in order to increase the proportion of cells lysed within the cellular material 104, increasing the yield of free subcellular components 106. The subcellular components 106 can then flow out of the temperature-regulated chamber 602 from a sample outlet 610. In certain embodiments, the first and second period of time can each independently be a period of time ranging from about 10 minutes to about 10 hours. In one example, the first period of time can be 1 hour and the second period of time can be 2 hours.

In certain embodiments, the temperature controlling device 600 can be controlled by the control unit 116. The control unit 116 can regulate flow of cellular material 104 through the sample inlet 608 and the sample outlet 610, the rate of heating and maximum temperature reached by the warming mechanism 606 and the rate of cooling and minimum temperature reached by the cooling mechanism 604.

### Photo Disruption Devices

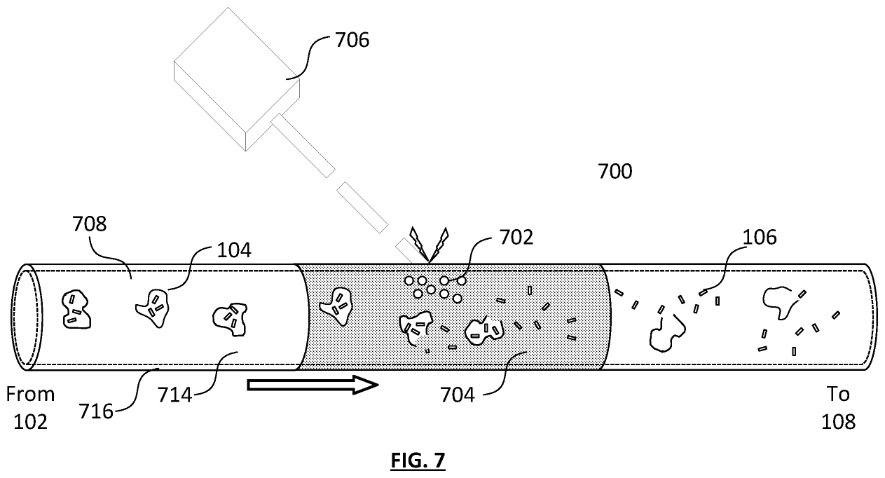

Referring now to FIG. 7, another embodiment of the cell disruption device 110 can be a photo disruption device 700. The photo disruption device 700 can disrupt cells through the use of short laser pulsed energy to create cavitation bubbles 702 within a medium including cellular material 104, whereby the cavitation bubbles 702 puncture cell membranes via high-speed fluidic flows and induced transient shear stress. The cavitation bubbles 702 can be formed by striking a thin film 704 including a coating or a plurality of nanoparticles with one or more short laser pulses 706 produced by a pulsed laser producing device 707. The cavitation bubble 702 pattern can be controlled by the thin film 704 coating or nanoparticle composition, structure or configuration. Additionally, the cavitation bubbles 702 can be controlled by altering the laser pulse 706 duration and energy level.

In one embodiment, the photo disruption device 700 includes a reservoir channel 708, including a first end 710, a second end 712, an internal lumen 714, and an external surface 716, and a laser source 706. The external surface 716 of the reservoir channel 708 can be coated with a thin film 704. In certain embodiments, the reservoir channel 708 can be composed of glass.

The laser 706 can be positioned to be directed at the reservoir channel 708 such that, when pulsed, the laser strikes the exterior 716 of the reservoir channel 708, causing the formation of a cavitation bubble within the reservoir channel 708. In certain embodiments, the laser can be focused at a point on the external surface 716 covered by the thin film 704, whereby the thin film 704 aids in absorbing and or transferring energy from the external surface 716 to the internal lumen 714.

A first end of the reservoir channel 708 can be in fluidic communication with the cellular material reservoir 102. The second end of the reservoir channel 708 can be in fluidic communication with the separation instrument 108.

In an exemplary procedure, cellular material 104 can be pumped from the cellular material reservoir 102, through the reservoir channel 708 as the pulsed laser strikes the thin film coating of the reservoir channel 708, creating cavitation bubbles 702. The cavitation bubbles 702 can lyse the cells within the cellular material 104 by exposing the cells to high shearing stresses and pressures as well as high energy electromagnetic radiation. Lysed cellular material 104 including subcellular components 106 can then flow from the reservoir channel 708, into the separation instrument 108.

The thin film 704 can include a metallic thin film and/or a plurality of nanoparticles (e.g., plasmonic nanoparticles). In certain embodiments, the thin film can comprise a material selected from the group consisting of a noble metal, a noble metal alloy, a noble metal nitride, a noble metal oxide, a transition metal, a transition metal alloy, a transition metal nitride, a transition metal oxide, a magnetic material, paramagnetic material, and a superparamagnetic material. In other embodiments, the thin film 704 can comprise a metal selected from the group consisting of gold and titanium. In certain embodiments, the thin film can be applied to the reservoir channel 708 through sputtered deposition.

The laser 706 can be any pulsed laser device capable of producing concentrated electromagnetic radiation capable of causing a cavitation bubble upon striking an absorbent material. The laser can produce radiation in the visible spectrum range (390 nm to 700 nm). In one example, the laser can be a 532 nm laser. The laser can be pulsed at a variety of rates from picoseconds to seconds but most preferably from about 0.1 ns to about 0.1 s. The laser can be positioned such that the beam encompasses the entire width of the reservoir channel 708 or only a portion thereof. In certain embodiments, the laser produces a cavitation bubble 702 capable of producing an instantaneous pressure within the reservoir channel 708 of about 1,500 Pa. In other embodiments, the laser illumination can produce laser illumination with an energy of about 500 J/m² to about 1,000 J/m², or any values in between. The laser 706 and the coating/nanoparticles 704 can be tuned/matched to each other in order to efficiently produce localized heating in response to the laser.

In certain embodiments, the laser 706 can be controlled by the control unit 116. The control unit 116 can also regulate flow through the reservoir channel 708.

### Projectile Force Devices

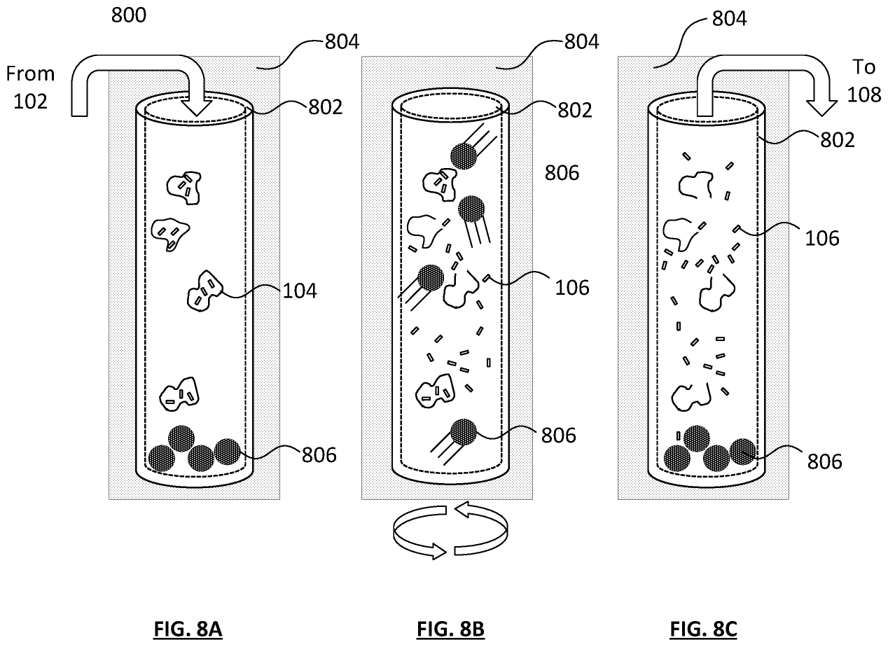

Referring now to FIGS. 8A-8C, another embodiment of the cell disruption device 110 can be a projectile force device 800 for the disruption of cell membranes using high-energy projectiles. The projectile force device can include one or more sample vessels 802 and an apparatus 804 adapted and configured to oscillate the one or more sample vessels 802. The sample vessels further include a plurality of grinding projectiles 806.

In certain embodiments, the apparatus 804 adapted and configured to oscillate the one or more sample vessels can be a centrifuge. The centrifuge can include a centrifugal motor attached to a fixture that is in turn attached to the one or more sample vessels such that the centrifugal motor rotates the tubes at an oblique angle at high speeds.

In another embodiment, the apparatus 804 can be a rotor or an impeller which is placed within a sample vessel 802 and rotated at high speed in order to oscillate the contents of the sample vessel 802.

In another embodiment, the apparatus 804 adapted and configured to oscillate the one or more sample vessels can be a vortex mixer.

The sample vessels can comprise grinding projectiles 806 made of one or more materials selected from metal, glass, silica, plastic and polymeric materials. In certain embodiments, the grinding projectiles 806 are beads. The beads can have any size or surface texture but are preferably smooth and spherically shaped. In one example, the grinding projectiles 806 can be glass beads having an average diameter of about 0.3 mm to 0.5 mm.

In certain embodiments, the sample vessels 802 can be standard laboratory sample vials or centrifuge vials composed of a material selected from the group consisting of glass, plastic and polymeric materials. In certain embodiments, the sample vessels 802 can be composed of silica, zirconia, polycarbonate or polyethylene. In certain embodiments, the number of sample vessels 802 is selected from the group consisting of 2 to 100, allowing for many samples to be processed simultaneously. The sample vessels 802 can be any reasonable volume which can be accommodated by the projectile force device 800. In a particular embodiment, the device includes 24 cylindrical, high-density polyethylene tubes with a volume of 2.0 mL.

In one example, the projectile force device 800 is a centrifuge including a high speed, brushless centrifugal motor attached to a fixture having a plurality of cylindrical tubes. Contained within each tube is a plurality of microbeads and a cellular material 104 sample taken from the cellular material reservoir 102. The centrifuge can then be made to rotate the tubes in high speed 3D motion. Microbeads within the tubes repeatedly collide with the cellular sample, resulting in high energy impacts that disrupt the membranes of the cells contained within the sample, releasing subcellular components 106. The free subcellular components can then be transferred to the separation instrument 108. In a particular embodiment of a centrifuge device, the device can be activated for approximately 30-40 seconds at an angular velocity of about 6 m/s.

The sample vessel 802 can also be a baffled container that includes a rotor apparatus. The cellular material 104 and a plurality of microbeads can be added to the baffled container, then the rotor apparatus can rotate at high speed, propelling the microbeads, resulting in high energy impacts between the microbeads and the cells. The impacts release the subcellular components 106, which can then be transferred to the separation instrument 108. In one example, the rotor can be operated in bursts with rest periods in between.

In some examples, the sample vessel(s) 802 can be kept at a temperature from about 0° C. to about 10° C.

In certain embodiments, the projectile force device can include an automated system adapted and configured to transfer cellular material 104 from the cellular material reservoir 102 to the one or more sample vessels and to transfer lysed cellular material 104 from the one or more sample vessels to the separation instrument 108. The automated system can be a robotic arm fitted with an array of pipettes or syringes adapted and configured to draw a specified volume of fluid and transfer the volume of fluid from one location to another.

### Chemical Disruption Device

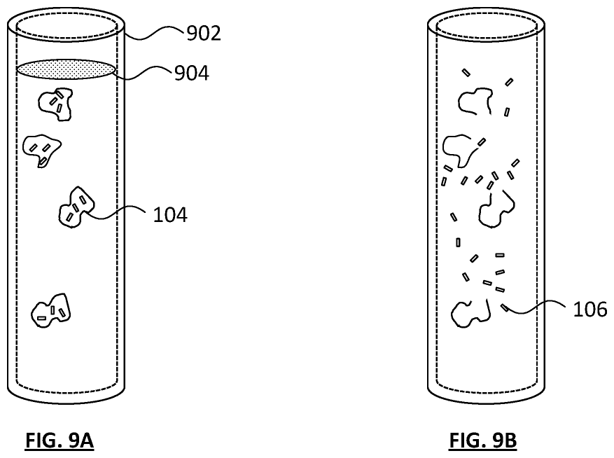

Referring now to FIGS. 9A and 9B another embodiment of the cell disruption device 110 can be a chemical disruption device for the disruption of cell membranes through chemical mechanisms. The device can include one or more sample vessels 902 adapted and configured for holding a cellular material 104 sample and a lysing agent 904. The device can further include an apparatus adapted and configured to oscillate the one or more sample vessels 902. The chemical disruption device can be operated by adding a cellular material 104 sample and a lysing agent 904 including one or more chemical lysing compounds to the one or more sample vessels and allowing the lysing agent 904 to disrupt the cells in the cellular material 104, releasing the subcellular components 106.

The cellular material 104 and the lysing agent 904 can be added to the sample vessels in any reasonable order. In certain embodiments, the cellular material 104 is added to the sample vessel before the lysing agent 904; in other embodiments, the cellular material 104 is added to the sample vessel after the lysing agent 904. In some embodiments, the lysing agent 904 can be dried onto an inner surface of the sample vessels 902.

In some embodiments, lysing and filtration can occur on a microfluidic device such as described in U.S. Patent Application Publication No. 2016-0215332.

### Subcellular Separation Devices

The invention provides an apparatus 100 for isolating one or more subcellular components from a cell, the apparatus comprising a separation instrument 108 configured to specifically isolate the subcellular components 106 based on one or more parameters.

In certain embodiments, the one or more parameters are selected from at least one of size, shape, density, charge/pH, magnetic attraction, spectral dispersion, spectral refraction, spectral diffraction, hydrophobicity, hydrophilicity, structure (presence or absence of a structural feature), and function (migration). The separation instrument 108 can induce at least one of a thermal change, a physical contact force (e.g., also shear contact force), an ultrasonic frequency, an osmotic change, a pressure change, a photothermal pulse, a magnetic field, an electromagnetic field, an electric field, and an electrical pulse in order to separate and isolate the subcellular components 106. In certain embodiments, the thermal change, the physical contact force, the ultrasonic frequency, the osmotic change, the pressure change, the photothermal pulse, the magnetic field, the electromagnetic field, the electric field, and the electrical pulse are generated as a gradient, a pulse, or a uniform wave.

In one embodiment, the separation instrument 108 separates the subcellular components 106 using a size gradient. The size gradient can include one or more membranes or filters, including microporous gels, beads, powders, meshes, microporous glasses and fibrous filter materials.

The pore size gradient can have variable pore sizes selected from the group consisting of less than about 50 μm, less than about 30 μm, less than about 15 μm, less than about 10 μm, less than about 9 μm, less than about 8 μm, less than about 7 μm, less than about 6 μm, less than about 5 μm, less than about 4 μm, less than about 3 μm, less than about 2 μm, and less than about 1 μm. In another embodiment, the size gradient has a size selected from the group consisting of the range of about 50 nm to about 50 μm, about 50 nm to about 15 μm, about 50 nm to about 10 μm, about 100 nm to about 5 μm, about 200 nm to about 5 μm, about 300 nm to about 5 μm, about 400 nm to about 5 μm, about 500 nm to about 5 μm, about 500 nm to about 4 μm, about 500 nm to about 3 μm, about 500 nm to about 2 μm, and about 500 nm to about 1 μm or any ranges in between.

The apparatus for isolating one or more subcellular components 100 can include two or more separation instruments 108 working in sequence. By combining multiple separation instruments 108 in sequence, the apparatus 100 can more completely isolate specific desired subcellular components 106 from the bulk cellular material 104.

The apparatus for isolating one or more subcellular components 100 can include two or more separation instruments 108 working in parallel. The use of two or more separation instruments 108 in parallel can increase throughput of the apparatus 100. In a preferred embodiment, the apparatus can include two or more separation instruments 108 which are identical or substantially identical operating in parallel, feeding into one or more subcellular component collection reservoirs 114.

### Imaging and Detection Devices

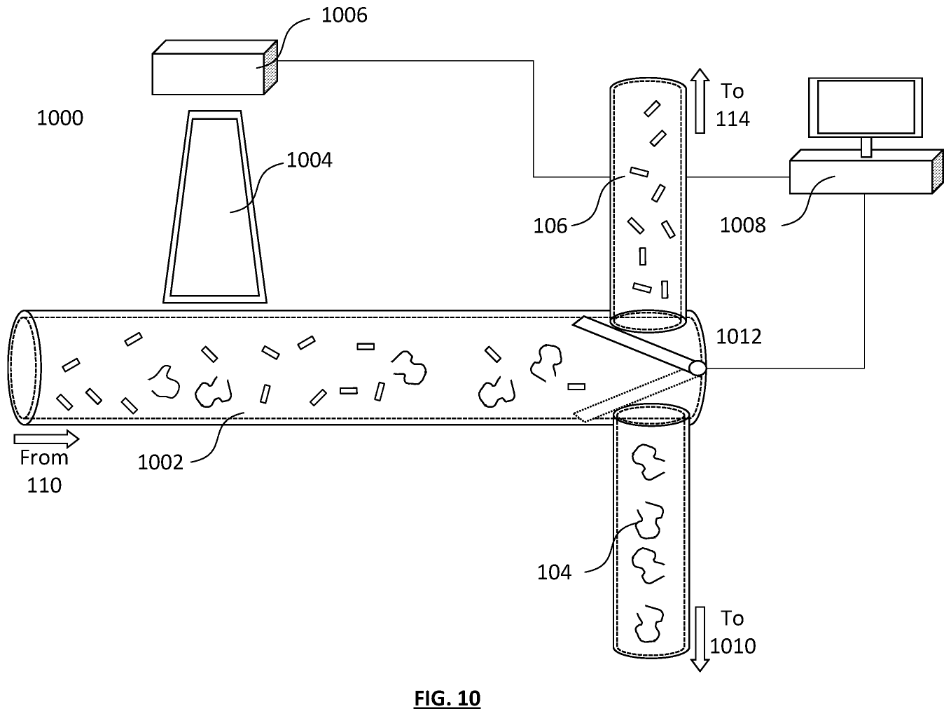

Referring now to FIG. 10, another embodiment of the separation instrument 108 provides an imaging system 1000 including a microfluidic reservoir 1002, a microscope 1004, a camera 1006, and an imaging computer 1008. The imaging system 1000 operates by analyzing subcellular components 106 flowing through the microfluidic reservoir 1002 by using a microscope 1004 connected to a camera 1006, which is in turn connected to a computer 1008. A computer algorithm identifies subcellular components 106 based on morphology and collects the desired subcellular components 106 in a subcellular component collection reservoir 114, which is in fluidic communication with the microfluidic reservoir 1002. The microfluidic reservoir can also be in fluidic communication with a waste reservoir 1010 which can collect any remaining, undesired cellular materials 104.

In certain embodiments, the microfluidic reservoir 1002 is created by photolithography on a substrate and reproduction using a moldable polymeric compound. The microfluidic channels can be made of polydimethylsiloxane (PDMS) “sandwiched” by transparent glass in order to create a closed, transparent channel to facilitate optical analysis by the microscope 1004 and camera 1006. The microfluidic reservoir 1002 can further comprise a physical gate 1012 which is in electronic communication with the imaging computer 1008. This physical gate 1012 can regulate flow into or away from the subcellular component collection reservoir 114. The gate 1012 can be selectively opened or closed by the imaging computer 1008 based on the morphology of the imaged subcellular components 106.

The main channel of the microfluidic reservoir 1002 should have a cross-sectional dimension larger than the subcellular components 106 which are intended to be sorted. In certain embodiments, the main channel has a cross-sectional dimension of about 1 μm to about 30 μm, or any cross-sectional dimension in between, most preferably, about 25 μm. In certain embodiments, the fluid flow rate through the main channel is about 10 mm/s, about 50 mm/s, 100 mm/s, about 200 mm/s, about 1,000 mm/s or any rate in between. The flow through the main channel can be driven by a pump with an adjustable flow rate.

The imaging system 1000 can include a camera 1006 attached to a microscope 1004. The microscope/camera 1004/1006 pairing can be used to actively monitor the subcellular components 106 as they pass through the microfluidic channel 1002. The microscope 1004 can be a confocal microscope. In certain embodiments, a picosecond-pulsed laser system generates two synchronized beams collinearly in an inverted confocal microscope in order to observe the subcellular components 106. The camera can then detect the epi- and forward-detected signal simultaneously as the subcellular components 106 pass through the channel. In one embodiment, the mean laser power can be about 21-28 mW at a wavelength of about 816-1064 nm. In certain embodiments, multiple simultaneous images at multiple wavelengths can be collected to aid in identifying individual subcellular components 106.

The camera 1004 can feed the imaging data to the imaging computer 1008 which can in turn run an image-analysis program to identify an established signal signature for the desired subcellular components 106 and can activate the physical gate 1012, diverting fluid flow towards the subcellular component collection reservoir 114. Once the signal signature is no longer observed, the computer directs the gate to close, directing the fluid flow away from the subcellular component collection reservoir 114 and towards the waste reservoir 1010, thereby separating the desired subcellular components 106 from the rest of the cellular materials 104.

In certain embodiments, the imaging system 1000 can be controlled by the control unit 116. The control unit 116 can include the imaging computer 1008 and can control the camera 1006, microscope 1004, physical gate 1012, and the flow of cellular material 104 through the microfluidic reservoir 1002.

### Filtration Devices

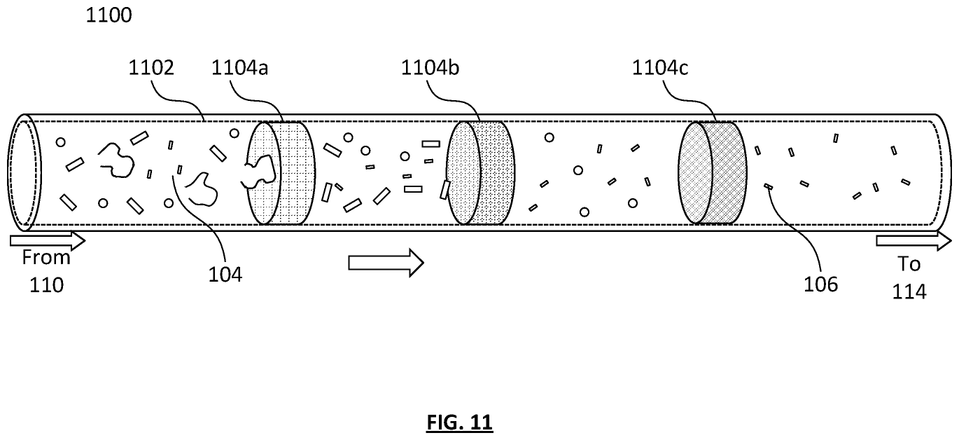

Referring now to FIG. 11, one embodiment of the separation instrument 108 is a filtration device 1100 capable of isolating subcellular components 106. The device 108 can include a microfluidic channel 1102 and one or more filters 1104a-c. The filtration device 1100 passes the subcellular components 106 through the one or more filters 1104a-c, removing undesired cellular material 104 and isolating desired subcellular components 106 by passing the subcellular components 106 into the subcellular component collection reservoir 114.

In certain embodiments, the sequential filters 1104a-c possess different pore sizes. In a preferred embodiment, the sequential filters 1104a-c possess decreasing pore sizes as the subcellular components 106 travel down the microfluidic channel 1102. The filters 1104a-c can have pore sizes of about 1 μm to about 50 μm or any pore size in between. In one embodiment, the filtration device 1100 comprises a microfluidic channel 1102 where homogenized cellular material 104 is passed through a series of three mesh filters, having pore sizes of 40 μm, 40 μm and 10 μm respectively, and into the subcellular component collection reservoir 114. In certain embodiments, the filters 1104a-c can comprise one or more filtering materials selected from the group consisting of mesh, microporous materials, beads and powders. The microporous materials can be microporous gels.

In one embodiment, the controlling unit 116 can regulate the flow of cellular material 104 through the filtration device 1100.

### Density Gradient

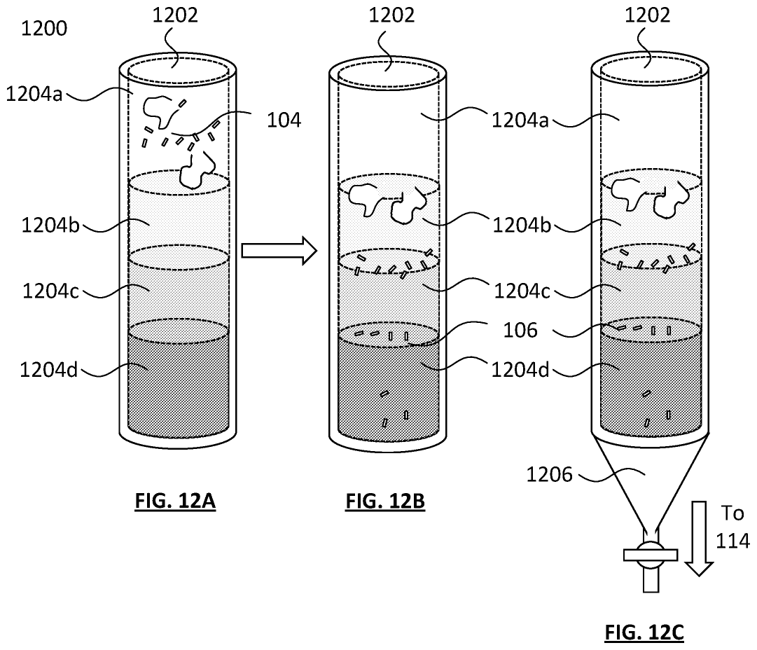

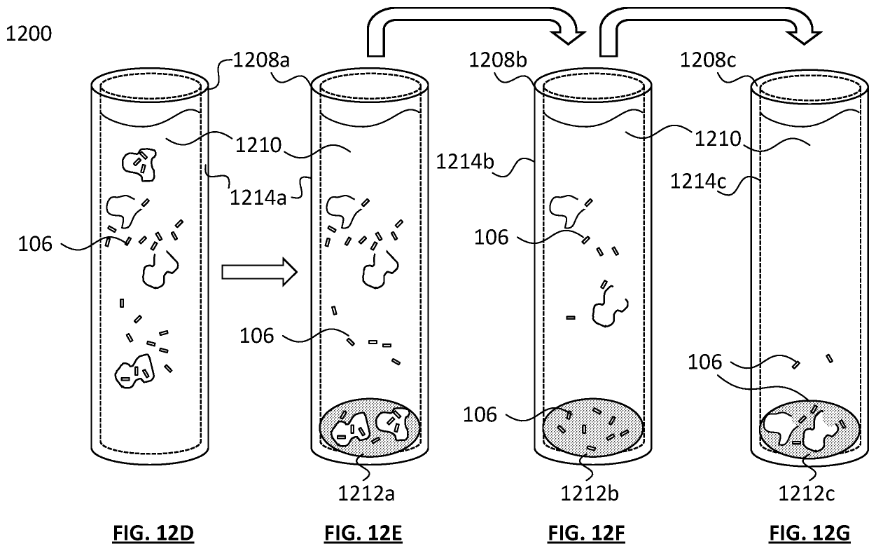

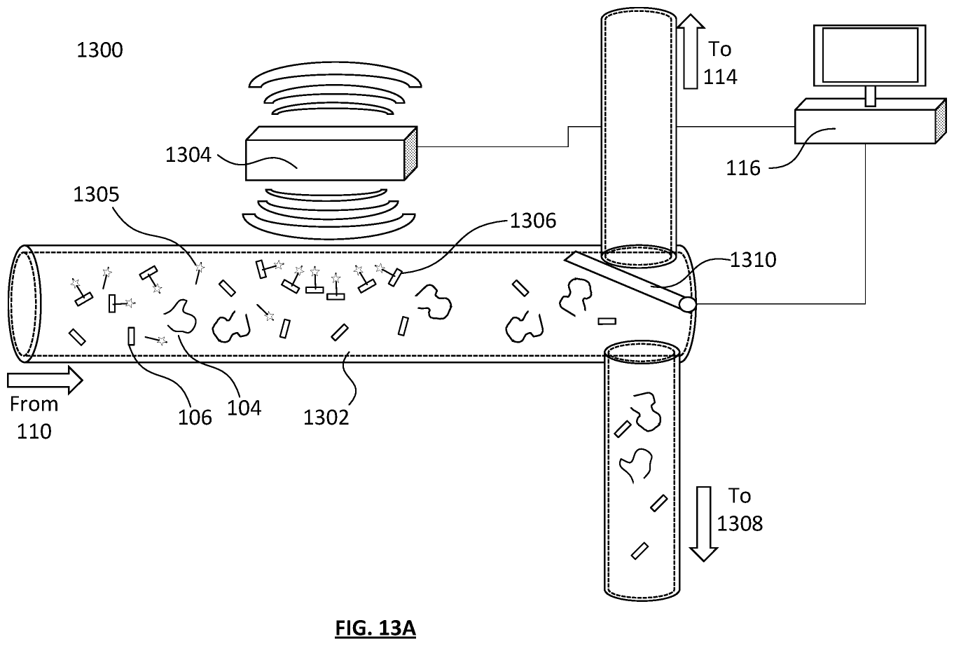

Referring now to FIGS. 12A-12G, one embodiment of the separation instrument 108 is a density gradient apparatus 1200 capable of isolating subcellular components 106 by allowing subcellular components to separate based on their specific densities.

In one embodiment, the density gradient apparatus 1200 comprises a reservoir 1202 comprising two or more fluids 1204a-d with different specific densities, separated into sequential layers. Subcellular components 106 separate based on their density relative to the density phases of the fluids 1204a-d.

The two or more fluids 1204a-d can be PERCOLL® (colloidal silica coated with polyvinylpyrrolidone) solutions with different concentrations. In other embodiments, the two or more fluids 1204a-d can be aqueous solutions of two or more biocompatible polymers, for example, dextran and polyethyleneglycol.

The subcellular components 106 can be separated by adding homogenized cellular material 104 to the reservoir 1202 comprising the two or more fluids 1204a-d. The subcellular components 106 can then diffuse into the fluids 1204a-d and arrive at the appropriate layer simply through natural gravitational pull. Alternatively, the reservoir 1202 can be centrifuged to increase the rate at which the subcellular components 106 separate. After separation, the layer containing the desired subcellular components can be isolated, for example by pipetting or decanting. In one embodiment, the reservoir 1202 can comprise an outlet spout 1206 located on the bottom of the apparatus, which allows for the sequential draining of the fluid layers 1204a-d, from densest to lightest, which can be fractioned off into different subcellular component collection reservoirs 114. In one embodiment, the reservoir can be centrifuged at 30,700 g at 4° C. for five minutes to force rapid separation of the subcellular components 106. Centrifuge speed and sedimentation temperature can be modified by a person of ordinary skill in the art to optimize separation of components.

In an alternative embodiment, the density gradient apparatus 1200 can include one or more reservoirs 1208a-c each including a fluid 1210. For example, a first reservoir 1208a comprising the subcellular components 106 can first be centrifuged at a low speed, whereby dense organelles and any remaining intact cells form a first pellet 1212a, leaving intermediate and low density organelles in the supernatant 1214a. The resulting supernatant 1214a can then be transferred to a second reservoir 1208b, which is then centrifuged at a higher speed, whereby intermediate density organelles form a pellet 1212b, leaving low density organelles in the supernatant 1214b. The resulting supernatant 1214b can then be transferred to a third reservoir 1208c, which is then centrifuged at an even higher speed, whereby low density organelles form a pellet 1212c, leaving only highly soluble, low density byproducts in the supernatant 1214c. This process can be repeated in sequence to create as many pelleted fractions as desired. In certain embodiments, the reservoir is centrifuged at about 1000 g, 10,000 g and 100,000 g in that order in order to form three pellets comprising different subcellular components 106 based on their specific densities and/or sedimentation velocities. In certain embodiments, each sequential centrifugation requires both a higher centrifuge speed and a longer centrifuge time in order to form the pellet. The pellet containing the desired subcellular components 106 can be collected and transferred to the subcellular component collection reservoirs 114.

### Magnetic Separation Devices

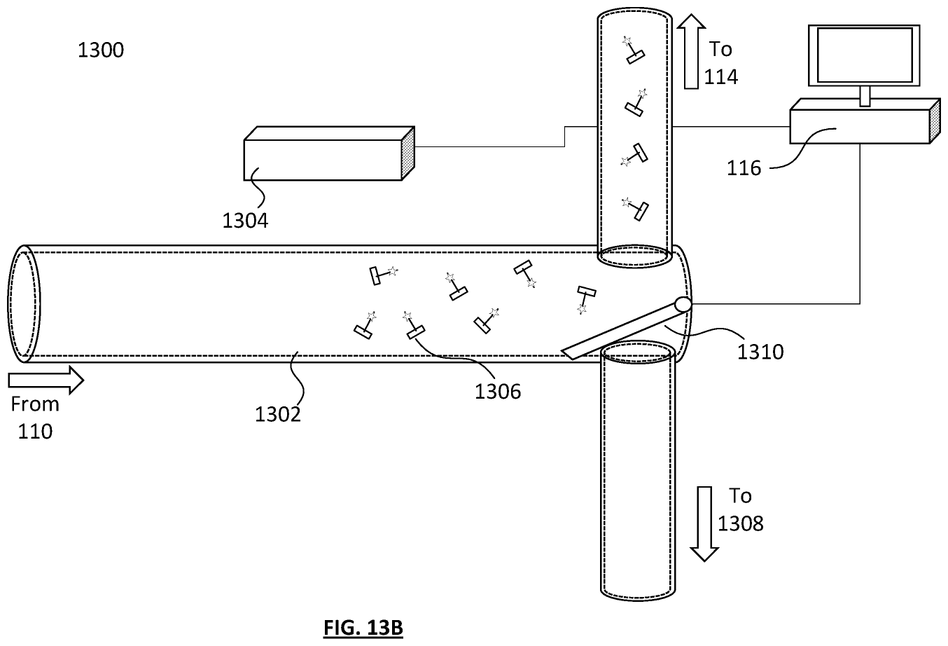

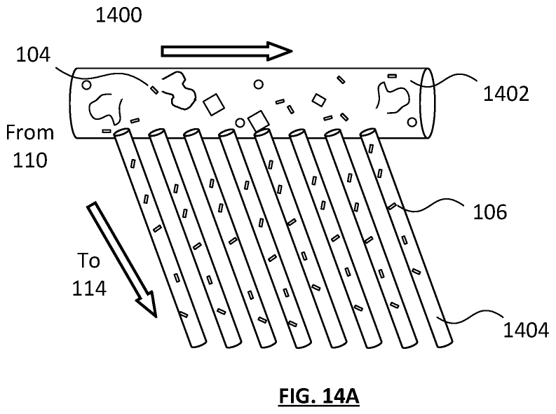

Referring now to FIGS. 13A and 13B, one embodiment of the separation instrument 108 is a magnetic separation device 1300 capable of isolating subcellular components 106 based on a magnetic or electromagnetic field. The magnetic separation device 1300 can include a microfluidic reservoir 1302 and a magnetic field generating device 1304 configured to generate a magnetic or electromagnetic gradient across the reservoir 1302. The magnetic separation device 1300 can utilize this magnetic or electromagnetic gradient by binding desired subcellular components 106 with a magnetically active label 1305 to generate a labelled subcellular component 1306. In one embodiment, the magnetically active label 1305 can be a magnetic bead conjugated to an antibody which can bind a protein on the surface of the desired subcellular component 106. The magnetically active label 1305 can be attracted to the generated magnetic or electromagnetic gradient, thereby inducing movement of the desired, labeled subcellular components 1306, allowing for separation of the desired subcellular components 1306 from the rest of the cellular material 104.

In certain embodiments, the magnetic separation device 1300 includes a microfluidic reservoir 1302 containing homogenized cellular material 104 containing subcellular components 106. The cellular material 104 is sequentially exposed to antibodies conjugated to magnetically active labels 1305 and wash buffers. The reservoir can further include a magnetic field generator 1304 that selectively generates a magnetic field. In one embodiment, the magnetic field is configured to attract magnetically labelled subcellular components 1306 and have no effect on unlabeled components. The microfluidic reservoir 1302 is then placed under a regulated fluid flow, whereby unlabeled subcellular components are washed out of the microfluidic reservoir 1302 and into a waste reservoir 1308, while the attracted magnetically labelled subcellular components 1306 are retained within the microfluidic reservoir 1302. After the unlabeled components 106 are removed, the magnetic field can be removed and the labelled components 1306 can be washed out of the microfluidic reservoir 1302 and into the subcellular component collection reservoir 114. One embodiment can further include a second magnetic field generator within the subcellular component collection reservoir 114 that can attract labelled subcellular components 1306 into the subcellular component collection reservoir 114 and away from the microfluidic reservoir 1302 and the waste reservoir 1308.

In certain embodiments, the magnetic separation device can include a physical gate 1310 that is adapted and configured to direct the flow of cellular material 104 towards the subcellular component collection reservoir 114 or the waste reservoir 1308. The physical gate 1310 and the magnetic field generator 1304 can be controlled by a controlling unit 116. The physical gate 1310 and the magnetic field generator 1304 can be coupled through the controlling unit 116 such that when the magnetic field generator 1304 is actively applying a magnetic field to the microfluidic reservoir 1302, attracting the labelled subcellular components 1306, the physical gate 1310 is oriented such that flow of cellular material 104 is directed towards the waste reservoir 1308 (See FIG. 13A) and when the magnetic field generator 1304 is not applying a magnetic field, the physical gate 1310 is oriented such that flow of cellular material 104 is directed towards the subcellular component collection reservoir 114 (See FIG. 13B). The controlling unit 116 can also regulate flow of cellular material through the microfluidic reservoir 1302.

### High-Throughput Size Retention Device

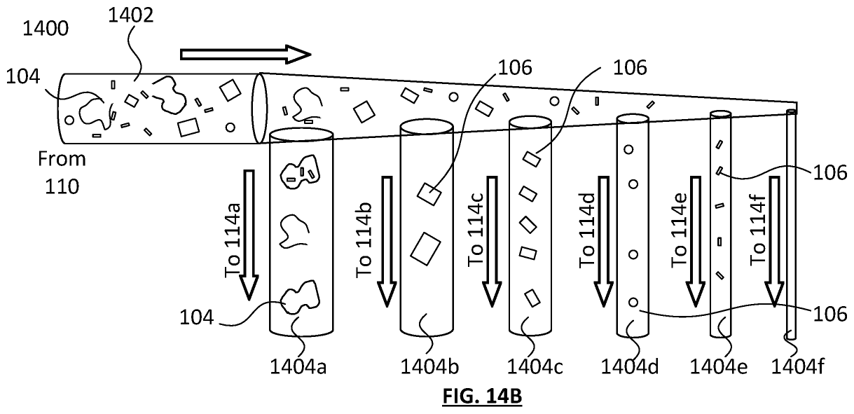

Referring now to FIGS. 14A and 14B, one embodiment of the separation instrument 108 is a high-throughput size retention device 1400 that utilizes micron and/or sub-micron restrictions in a nanofluidic or microfluidic device to isolate subcellular components 106 from homogenized cellular material 104 in a high throughput fashion based on relative size of the subcellular components 106.

The size retention device 1400 includes a microfluidic channel 1402 and a series of branched nanoscale channels 1404 in fluidic communication with the microfluidic channel 1402. The branched nanoscale channels 1404 can be of different cross-sectional diameters or of the same cross-sectional diameter. The branched nanoscale channels 1404 can be joined with the microfluidic channel 1402 at different locations along the microfluidic channel 1402. In some embodiments, the microfluidic channel 1402 can have a consistent cross-sectional diameter or it can be tapered such that it becomes narrower or wider, having a larger or smaller cross-sectional diameter.

In one embodiment, the microfluidic channel 1402 is joined with a series of two or more nanoscale channels 1404 of identical cross-sectional diameter. The microfluidic channel 1402 can have a cross-sectional diameter of sufficient size as to allow the free flow of the homogenized cellular material 104. In certain embodiments, the microfluidic channel 1402 has a cross-sectional diameter from about 10 μm to about 100 μm or any diameter in between. The nanoscale channels 1404 can have a cross-sectional diameter equal to or greater than the width of the desired subcellular components 106. In certain instances, the cross-sectional diameter of the nanoscale channels is about 0.2 μm to about 2.0 μm wider than the desired subcellular components. In other embodiments, the nanoscale channels 1404 have a cross-sectional diameter of about 0.4 μm to about 3.0 μm or any diameter in between. In one embodiment, the nanoscale channels 1404 can have an oblong or rectangular cross section with a minimum cross-sectional diameter of about 0.45 μm to about 0.75 μm and a maximum cross-sectional diameter of about 2 μm. A fluid containing homogenized cellular material 104 can be flowed through the microfluidic channel 1402 and past the series of nanoscale channels 1404. As the cellular material 104 flows past the nanoscale channels 1404, subcellular components 106 of the desired size can flow into the nanoscale channels 1404 while larger subcellular components remain in the bulk cellular material 104 in the microfluidic channel 1402. The remaining cellular material 104 can flow from the microfluidic channel 1402 into a waste reservoir 1406 or can be recirculated past the nanoscale channels 1404 in order to allow more of the desired subcellular components 106 to pass into the nanoscale channels 1404. The nanoscale channels 1404 in turn can be in fluidic communication with one or more subcellular component collection reservoirs 114 where the desired subcellular components 106 can be collected.

In an alternative embodiment, the nanoscale channels 1404 can be of varied widths allowing for selective fractionation of subcellular components 106. In certain embodiments, the microfluidic channel 1402 is tapered, preventing larger subcellular components 106 from progressing down the microfluidic channel 1402 and forcing them to divert into a nanoscale channel 1404a with a sufficient cross-sectional diameter to accommodate the size of the subcellular component 106. Smaller subcellular components 106 can continue further down the tapered microfluidic channel 1402 until a point where they are too large to proceed further and are forced to divert into a smaller nanoscale channel 1404b. Each nanoscale channel 1404a-f can be in fluidic communication with a different subcellular component collection reservoir 114a-f. By utilizing nanoscale channels with progressively smaller cross-sectional diameters, the high-throughput size retention device can isolate subcellular components 106 of varying sizes.

In certain embodiments, the high-throughput size retention device 1400 can be fabricated in polydimethylsiloxane (PDMS) using photolithography of a positive photoresist on a silicon substrate. To create an enclosed space for fluid flow, the PDMS portion is bonded to a glass surface. The cellular material 104 can be passed through the channels using a pump with a variable flow rate. In one embodiment, the fluid containing the cellular material 104 can be pumped at a rate of 10 μL/hour for 2 minutes.

In one embodiment, a controlling unit 116 can regulate flow of cellular material through the microfluidic channel 1402.

In another embodiment, separation can be achieved by manipulating microfluidic flow (e.g., through placement of posts and other structures) as described in Daniel R. Gossett et al., “Label-free cell separation and sorting in microfluidic systems”, 397 Anal. Bioanal. Chem. 3249-67 (2010).

### Methods of Isolating Subcellular Components

The invention further includes methods of isolating subcellular components 106 from cellular material 104 using the apparatus 100 of the invention.

In certain embodiments, the method includes: disrupting cellular material 104 comprising intact cells using a cell disruption device 110 according to an embodiment of the invention; transferring the disrupted cellular material 104 comprising free subcellular components 106 to a separation instrument 108 according to an embodiment of the invention and allowing the separation instrument 108 to isolate the desired subcellular components; and collecting the isolated subcellular components 106.

Implementation in Computer-Readable Media and/or Hardware

The methods described herein can be readily implemented in software that can be stored in computer-readable media for execution by a computer processor. For example, the computer-readable media can be volatile memory (e.g., random access memory and the like), non-volatile memory (e.g., read-only memory, hard disks, floppy disks, magnetic tape, optical discs, paper tape, punch cards, and the like).

Additionally or alternatively, the methods described herein can be implemented in computer hardware such as an application-specific integrated circuit (ASIC).

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Example 1: Tissue Homogenizer

This example describes a device that disrupts tissues and cells through homogenization, without damaging subcellular components. The device includes a tubular container made of glass. The tubular container includes a pestle, made of Teflon, mounted to a shaft and a motor. The pestle has a grooved outer surface, approximately 0.125 inches in depth, and is located in close proximity (0.002 inches) to the inside surface of the tubular container. Tissue is added into the tubular container, and a rotating pestle moves at a rate of at least 200 revolutions per minute (RPM). The motion of the pestle within the tube homogenizes the tissue through shear force, resulting in the breakdown of connective tissue, proteins, and cell membranes. The pestle rotation rate is steadily increased to 1000 RPM over a period of five minutes to gradually increase the degree of tissue homogenization.

### Example 2: Microfluidic Cell or Tissue Disruptor

This example describes a microfluidic-based device that disrupts cellular membranes through physical force. A reservoir of cells is connected to a series of microfluidic channels with a small diameter, such that cells are constricted when pumped through the channels, resulting in temporary or permanent loss of cell membrane integrity due to pressure and shear stress.

A microfluidic system includes microfluidic channels, containing one or more constrictions, etched onto a silicon chip and sealed by a layer of Pyrex glass. The channels are one cm in length. The width and depth of each constriction ranges from 4-8 μm and 10-50 μm, respectively. The throughput rate is about 20,000 cells/s. Pressure from the pump and shear stress deforms the cells to move through the microfluidic channels and constrictions. Each constriction is less than ˜50% diameter of the cell, but larger than the diameter of the desired subcellular component. A parallel channel design increases throughput, while insuring uniform treatment of cells, because any clogging or defects in one channel does not affect the flow speed in a neighboring channel. Prior to use, the device is connected to a steel interface that connects the inlet and outlet reservoirs to the microfluidic system. A mixture of cells and the desired delivery material are then placed into the inlet reservoir and Teflon tubing is attached at the inlet. A pressure regulator is then used to adjust the pressure at the inlet reservoir and drive the cells through the device. Cellular material is collected in the outlet reservoir.

### Example 3: Sonicator

This example describes a device for the disruption of cells using energy from ultrasound waves. Tissue, approximately 500 grams, is placed in a polyethylene reservoir along with 100 ML of collagenase solution. All air is removed from the reservoir via an outlet, and the reservoir is enclosed in a water bath/sonication device. The water bath is conditioned to 37° C. The tissue is sonicated at a frequency of 43 kHz for 20 minutes, at a power of 0.9 watt/cm². The sonicated tissue is pumped from the reservoir through a stainless steel screen (nominal mesh size of 350-500 μm) into a channel. Next, the tissue is pumped through a second screen or filter (nominal mesh size of 20-50 μm) and cellular material is collected in a secondary reservoir.

### Example 4: Gas-Cavitation Device

This example describes a device for disruption of cells or tissue using gas cavitation based on differential gas pressure. The device dissolves nitrogen within cells under high pressure within a pressure vessel, then rapidly releases pressure. This causes nitrogen to come out of solution. Gas bubbles increase in size, stretching and ultimately disrupting cell membranes.

Tissue is placed in a chamber of a cell disruption device. The device is 920 mL in volume, accommodating a sample size of 600 mL. The cylindrical chamber is 3.75 inches in diameter and 5.10 inches in height. A pressure cap with a rubber gasket seal, in the closed position, is placed on the cell disruption chamber and connected to a nitrogen source through a valve mounted on the cap. With the pressure cap closed, nitrogen is pumped into solution at a rate of 100 mL/min. The pressure cap is simultaneously opened to pressurize the inner chamber to 1000 psi. After pressurization, the pressure cap and nitrogen tank are closed, in sequential order. Then, pressure is rapidly decreased to atmospheric pressure (14.7 PSI). A collection valve at the base of the cell disruption device is opened and the lysed cells are collected in a reservoir.

### Example 5: Temperature-Controlled Device

This example describes a device used to sequentially freeze and thaw cells or tissue to disrupt cellular integrity.

The device contains a temperature-regulated chamber with a cooling mechanism, capable of driving the temperature to −20° C. Additionally, the device contains a warming mechanism capable of driving the temperature of 37° C. A cell suspension is placed in the chamber. The device cools the temperature in the chamber to −20° C. over a period of one hour, causing the cells to swell and ultimately break as ice crystals form during the freezing process. Next, the device gradually warms the temperature in the chamber to 37° C. over a period of two hours, causing the cells to contract during thawing. This process is repeated two more times to result in cell lysis.

### Example 6: Photo-Disruption Device

This example describes a device for the disruption of cells through the induction of membrane openings through the use of light and pressure. A metallic nanostructure converts short laser pulsed energy to explosive vapor bubbles that rapidly puncture the cell membrane via high-speed fluidic flows and induced transient shear stress. The cavitation bubble pattern is controlled by the metallic structure configuration and laser pulse duration and energy level. In this device, a glass reservoir channel is coated with a 100 nm titanium thin film on the surfaces of the channel using a sputterer deposition system. The glass reservoir is connected to an external pressure source and a 532 nm nanosecond pulsed laser. The laser is positioned to encompass the width of the channel, controlled by a microscope epifluorescence port.

Cells are pumped through the reservoir channel and simultaneously exposed to pressure of 15 hPa and laser illumination of 883 J/m² for 0.1 seconds, resulting in cavitation bubbles to open the cell membranes. Lysed cellular material is collected in a secondary reservoir.

### Example 7: Projectile-Force Device

This example describes a device for the disruption of cell membranes using high-energy projectiles. The device includes a high speed, brushless centrifugal motor attached to a fixture having 24 cylindrical HDPE tubes, 2.0 mL in volume. Each tube has zirconium microbeads, 1.5 mm in diameter. The motor rotates the tubes at an oblique angle, such that polymeric beads move idiosyncratically in three dimensions at high speed.

A cellular sample is added to each of the tubes within the device, then subjected to high speed 3D motion. Microbeads within the tubes repeatedly collide with the sample, resulting in high energy impact to disrupt cell membranes. The device is activated for 35 seconds at an angular velocity of 6 m/s. Cellular material subsequently aspirated from the sample tubes.

### Example 8: Chemical-Disruption Device

This example describes a device to promote cell lysis through a chemical mechanism. The device includes a fixture to capture a standard 96-well cell culture plate connected to a nutating shaker. It also includes an automated, moveable manifold with 12×0.1 mm-diameter nozzles connected to a fluid reservoir. The manifold dispenses a controlled amount of fluid into the plate, 12 wells at a time.

100 μL cellular samples are distributed into a standard 96-well plate. The reservoir contains 0.1% TRITON™ X-100, a cocktail of detergents for the disruption of lipid bilayer membranes. The device pumps 100 μL of detergent into each well of the plate, then gently agitates the plate for 30 minutes at room temperature. This action results in cell lysis. The cellular material is removed from each of the wells.

### Example 9: Imaging and Detection Methods Using a Computer, Camera, and Microscope

In this example, subcellular components are identified and isolated using an image analysis-enabled device. The device includes a microfluidic channel that analyzes and isolates subcellular components from the cellular material following disruption of the cell membrane, for example obtained by any of Examples 1-6. Cellular material is analyzed by a microscope connected to a camera, which in turn is connected to a computer. A computer algorithm identifies subcellular components based on their morphology and collects the subcellular components in a final reservoir.

The imaging system includes the following components: a microfluidic reservoir, a microscope/camera for visualization of objects within the reservoir, and a computer for real-time image analysis. The microfluidic channel is created by photolithography of a silicon substrate, reproduced using PDMS. Glass is bonded to either side of the PDMS structure, creating a closed, transparent channel to facilitate optical analysis. The main channel is 25 μm in diameter, with a fluid flow rate of 100 mm/s driven by a pump. The main channel is also connected to a collection reservoir, which opens selectively when a physical gate is activated by the computer.

A microscope is used to actively monitor the cellular material as it passes through the microfluidic channel. The instrument includes a confocal microscope. A picosecond-pulsed laser system generates two synchronized beams collinearly aligned in an inverted confocal microscope. The mean laser power is 28 mW at 816 nm. The epi- and forward-detected signal are measured simultaneously with a camera/detector as the cellular material passes through the channel.

A computer image-analysis program identifies an established signal signature for the subcellular components, and activates a physical gate, diverting the fluid flow to the collection reservoir. After the established signal signature is no longer visible, the gate is closed and fluid flow continues to waste. Subcellular components are selectively captured in the collection reservoir.

### Example 10: Filtration Device

In this example, subcellular components are isolated based on a size gradient using a filtration device. The device includes a microfluidic channel that isolates subcellular components from the cellular material following disruption of the cell membrane, for example obtained by any of Examples 1-6. The device includes sequential filters of decreasing size in fluid connection with one another. The instrument passes the subcellular components through the filters removing non-target cellular material, and isolating target subcellular components.

The filtration device includes a microfluidic reservoir, where the cellular material is passed through a 40 μm (pore size) mesh filter, a second 40 μm mesh filter, and a final 10 μm mesh filter. Finally, the filtrate is passed into a collection reservoir.

The collection reservoir is placed in a centrifuge and spun at 9000×g for 10 minutes at 4° C. to concentrate the subcellular components.

### Example 11: Density Gradient

In this example, subcellular components are isolated from cellular material, for example obtained by any of Examples 1-6, by a device having a density gradient and configured to rotate at variable speeds. A reservoir holds multiple fluids of specific densities, oriented sequentially. Subcellular components are differentially separated based on their density relative to density phases of the other solutions.

The device includes a density gradient having Percoll solutions at 40%, 23%, and 15% concentrations in a translucent round-bottomed polycarbonate or pollayallomer reservoir. Percoll is composed of colloidal silica coated with polyvinylpyrrolidone, and is commonly used for the isolation of cellular components.

The cellular material is slowly layered into the percoll gradient via an inlet in the reservoir. Although it is theoretically possible to separate subcellular components by gravity, high-speed centrifugation of the reservoir increases throughput. The reservoir is centrifuged at 30,700 g at 4° C. for five minutes to create three distinct bands of material within the vial. Using a glass Pasteur pipet, the band containing the desired subcellular components is removed from the device.

### Example 12: Magnetic Separation Device

In this example, subcellular components are isolated from cellular material, for example obtained by any of Examples 1-6, based on a magnetic or electromagnetic field. The device includes a microfluidic reservoir with electrical circuitry configured to generate a magnetic or electromagnetic gradient across the reservoir. Magnetic beads conjugated to antibodies bind a subcellular component protein and are differentially attracted to the magnetic or electromagnetic field.

The magnetic separation device includes a microfluidic reservoir, where the cellular material is sequentially exposed to antibodies and wash buffers. The reservoir has a regulated fluid flow rate of 0-100 mm/s driven by a computer-controlled pump. The reservoir is connected to a magnetic field generator, which opens selectively generates a magnetic pulse/field/gradient that is activated by the computer. The reservoir is also connected to a collection reservoir.

Activation of the magnetic field generator establishes a magnetic gradient that retains the subcellular components in the reservoir. Deactivation of the magnetic field generator allows the magnetic bead-labeled subcellular components to be diverted in the fluid flow to the collection reservoir.

The collection reservoir is placed in a centrifuge and spun at 9000×g for 10 minutes at 4° C. to concentrate the subcellular components.

### Example 13: High-Throughput Size Retention Device

This example describes the use of a separation instrument with sub-micron constrictions in a nanofluidic/microfluidic device to isolate subcellular components from cellular material, for example obtained by any of Examples 1-6, in a high throughput fashion based on relative size.

In this example, cellular material is delivered to a surface with a chamber having a series of branched, nanoscale channels that support fluid flow. At each branch, the nanoscale channels diverge, with one side of the branch being a “trapping” channel and the other side of the branch being a “waste” channel. The trapping channel sequentially decreases in cross-sectional diameter until only subcellular components of particular sizes are passed through the trapping channel and all other cellular material and debris is diverted to waste channels.

The device is fabricated in polydimethylsiloxane (PDMS) using photolithography of a positive photoresist on a silicon substrate. To create an enclosed space for fluid flow, the PDMS portion is bonded to a glass surface. The waste nanochannel cross-sectional diameter ranges from 250-1000 nm in length and ˜10-80 μm in width. Each trapping channel has a cross-sectional dimension about 2 μm in one direction and a cross-sectional dimension between about 0.45 and about 0.75 μm in a second direction. The most-downstream trapping channel is designed to selectively capture the subcellular components. The width is 2 μm, which is larger than the width of the desired subcellular components (0.2-1.2 μm). The height of the channels (0.45-0.75 μm) is almost equal to the average diameter of the desired subcellular components.

Cellular material is added to the holding reservoir, then passed through the channels using a pump. All channels are pumped at a rate of 10 μL/hour for 2 minutes. Subcellular components are selectively captured in a downstream reservoir.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

### APPENDIX

### Definitions

As used herein, a “cell membrane” refers to a membrane derived from a cell, e.g., a source cell or a target cell.

As used herein, a “chondrisome” is a subcellular apparatus derived and isolated or purified from the mitochondrial network of a natural cell or tissue source. A “chondrisome preparation” has bioactivity (can interact with, or have an effect on, a cell or tissue) and/or pharmaceutical activity.

As used herein, a chondrisome preparation described herein is “stable” when it maintains a predefined threshold level of its activity and structure over a defined period of time. In some embodiments, one or more (2 or more, 3 or more, 4 or more, 5 or more) structural and/or functional characteristics of a chondrisome preparation described can be used as defining metrics of stability for chondrisome preparations described herein. These metrics, whose assay protocols are outlined herein, are determined subsequent to preparation and prior to storage (e.g., at 4 C, 0 C, −4 C, −20 C, −80 C) and following removal from storage. The characteristic of the preparation should not change by more than 95%, 90%, 85%, 80%, 75%, 60%, 50% (e.g., no more than 40%, 35%, 30%, 25%, 20%, 15%, 10%, 5%) over the course of 1, 2, 5, 8, 12, 24, 36, or 48 hours, 3 days, 7 days, 14 days, 21 days, 30 days, 60 days, 90 days, 4 months, 6 months, 9 months, a year or more of storage. In some embodiments, the characteristic of the chondrisome preparation described herein should not have changed by more than 50% (e.g., no more than 40%, 35%, 30%, 25%, 20%, 15%, 10%, 5%) over the course of 1, 2, 5, 8, 12, 24, 36, or 48 hours of storage. In some embodiments, the characteristic of the chondrisome preparation described herein should not change by more than 50% (e.g., no more than 40%, 35%, 30%, 25%, 20%, 15%, 10%, 5%) over the course of 1, 2, 5, 8, 12, 24, 36, or 48 hours, 3 days, 7 days, 14 days, 21 days, 30 days, 60 days, 90 days, 4 months, 6 months, 9 months, a year or more of storage.

As used herein, “cytobiologic” refers to a portion of a cell that comprises a lumen and a cell membrane, or a cell having partial or complete nuclear inactivation. In some embodiments, the cytobiologic comprises one or more of a cytoskeleton component, an organelle, and a ribosome. In embodiments, the cytobiologic is an enucleated cell, a microvesicle, or a cell ghost.

As used herein, “cytosol” refers to the aqueous component of the cytoplasm of a cell. The cytosol may comprise proteins, RNA, metabolites, and ions.

An “exogenous agent” as used herein, refers to an agent that: i) does not naturally exist, such as a protein that has a sequence that is altered (e.g., by insertion, deletion, or substitution) relative to an endogenous protein, or ii) does not naturally occur in the naturally occurring source cell of the fusosome in which the exogenous agent is disposed.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., the lumen of the fusosome and a cytoplasm of a target cell).

As used herein, “fusogen binding partner” refers to an agent or molecule that interacts with a fusogen to facilitate fusion between two membranes.

As used herein, “fusosome” refers to a membrane enclosed preparation and a fusogen that interacts with the amphipathic lipid bilayer.

As used herein, “fusosome composition” refers to a composition comprising one or more fusosomes.

As used herein, “locally” or “local administration” means administration at a particular site of the body intended for a local effect. Examples of local administration include epicutaneous, inhalational, intra-articular, intrathecal, intravaginal, intravitreal, intrauterine, intra-lesional administration, lymph node administration, intratumoral administration, administration to a fat tissue or mucous membrane of the subject, wherein the administration is intended to have a local effect. Local administration may also include perfusion of the preparation into a target tissue. For example, a preparation described herein may be delivered locally to the cardiac tissue (i.e., myocardium, pericardium, or endocardium) by direct intracoronary injection, or by standard percutaneous catheter based methods or by perfusion into the cardiac tissue. In another example, the preparation is infused into the brain or cerebrospinal fluid using standard methods. In another example, the preparation is directly injected into adipose tissue of a subject.

As used herein, “membrane enclosed preparation” refers to a bilayer of amphipathic lipids enclosing a cargo in a lumen or cavity. In some embodiments, the cargo is exogenous to the lumen or cavity. In other embodiments, the cargo is endogenous to the lumen or cavity, e.g., endogenous to a source cell.

As used herein, “mitochondrial biogenesis” denotes the process of increasing biomass of mitochondria. Mitochondrial biogenesis includes increasing the number and/or size of mitochondria in a cell.

As used herein, the term “purified” means altered or removed from the natural state. For example, a cell or cell fragment naturally present in a living animal is not “purified,” but the same cell or cell fragment partially or completely separated from the coexisting materials of its natural state is “purified.” A purified fusosome composition can exist in substantially pure form, or can exist in a non-native environment such as, for example, a culture medium such as a culture medium comprising cells.

As used herein, a “source cell” refers to a cell from which a fusosome is derived.

As used herein, a “subcellular component” is a subcellular apparatus derived and isolated or purified from a natural cell or tissue source.

### Fusosomes

In some aspects, the fusosome compositions and methods described herein comprise membrane enclosed preparations, e.g., naturally derived or engineered lipid membranes, comprising a fusogen. In some aspects, the disclosure provides a portion of a non-plant cell, e.g., a mammalian cell, or derivative thereof (e.g., a mitochondrion, a chondrisome, an organelle, or an enucleated cell), which comprises a fusogen, e.g., protein, lipid and chemical fusogens.

### Fusogens

In some embodiments, the fusosome described herein (e.g., a liposome, a vesicle, a portion of a cell) includes one or more fusogens, e.g., to facilitate the fusion of the fusosome to a membrane, e.g., a cell membrane. Also, these compositions may include surface modifications made during or after synthesis to include one or more fusogens, e.g., fusogens may be complementary to a target cell.

In some embodiments, the fusosomes comprise one or more fusogens on their exterior surface to target a specific cell or tissue type (e.g., cardiomyocytes). Fusogens include, without limitation, protein based, lipid based, and chemical based fusogens. The fusogen may bind a partner on a target cells' surface. In some embodiments, the fusosome comprising the fusogen will integrate the membrane into a lipid bilayer of a target cell.

In some embodiments, one or more of the fusogens described herein may be included in the fusosome.

### Protein Fusogens

In some embodiments, the fusogen is a protein fusogen, e.g., a mammalian protein or a homologue of a mammalian protein (e.g., having 50%, 60%, 70%, 80%, 85%, 90%, 95%, 96%, 97%, 98%, 99%, or greater identity), a non-mammalian protein such as a viral protein, a native protein or a derivative of a native protein, a synthetic protein, a fragment thereof, a protein fusion comprising one or more of the fusogens or fragments, and any combination thereof.

### Mammalian Proteins

In some embodiments, the fusogen may include a mammalian protein. Examples of mammalian fusogens may include, but are not limited to, a SNARE family protein such as vSNAREs and tSNAREs, a syncytin protein such as Syncytin-1 (DOI: 10.1128/JVI.76.13.6442-6452.2002), and Syncytin-2, myomaker (biorxiv.org/content/early/2017/04/02/123158, doi.org/10.1101/123158, doi: 10.1096/fj.201600945R, doi:10.1038/nature12343), myomixer (www.nature.com/nature/journal/v499/n7458/full/nature12343.html, doi:10.1038/nature12343), myomerger (science.sciencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in U.S. Pat. No. 6,099,857A), a gap junction protein such as connexin 43, connexin 40, connexin 45, connexin 32 or connexin 37 (e.g., as disclosed in US 2007/0224176, Hap2, any protein capable of inducing syncytium formation between heterologous cells (see Table 2), any protein with fusogen properties (see Table 3), a homologue thereof, a fragment thereof, a variant thereof, and a protein fusion comprising one or more proteins or fragments thereof. In some embodiments, the fusogen is encoded by a human endogenous retroviral element (hERV) found in the human genome. Additional exemplary fusogens are disclosed in U.S. Pat. No. 6,099,857A and US 2007/0224176, the entire contents of which are hereby incorporated by reference.

### Non-Mammalian Proteins

In some embodiments, the fusogen may include a non-mammalian protein, e.g., a viral protein. In some embodiments, a viral fusogen is a Class I viral membrane fusion protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV).

In some embodiments, Class III viral membrane fusion proteins include, but are not limited to, rhabdovirus G (e.g., fusogenic protein G of the Vesicular Stomatatis Virus (VSV-G)), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), and Borna disease virus (BDV) glycoprotein (BDV G).

Examples of other viral fusogens, e.g., membrane glycoproteins and viral fusion proteins, include, but are not limited to: viral syncytia proteins such as influenza hemagglutinin (HA) or mutants, or fusion proteins thereof; human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukaemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus; canine distemper virus, Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukaemia virus and Mason Pfizer monkey virus; mumps virus hemagglutinin neuraminidase, and glyoproteins F1 and F2; membrane glycoproteins from Venezuelan equine encephalomyelitis; paramyxovirus F protein; SIV gp160 protein; Ebola virus G protein; or Sendai virus fusion protein, or a homologue thereof, a fragment thereof, a variant thereof, and a protein fusion comprising one or more proteins or fragments thereof.

Non-mammalian fusogens include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic postfusion conformation with a signature trimer of α-helical hairpins with a central coiled-coil structure. Class I viral fusion proteins include proteins having a central postfusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of β-sheets forming an elongated ectodomain that refolds to result in a trimer of hairpins. In embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises a helices (e.g., forming a six-helix bundle to fold back the protein as with class I viral fusogens), and β sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi:10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi:10.1016/j.devce1.2007.12.008).

Additional exemplary fusogens are disclosed in U.S. Pat. No. 9,695,446, US 2004/0028687, U.S. Pat. Nos. 6,416,997, 7,329,807, US 2017/0112773, US 2009/0202622, WO 2006/027202, and US 2004/0009604, the entire contents of all of which are hereby incorporated by reference.

### Other Proteins

In some embodiments, the fusogen may include a pH dependent (e.g., as in cases of ischemic injury) protein, a homologue thereof, a fragment thereof, and a protein fusion comprising one or more proteins or fragments thereof. Fusogens may mediate membrane fusion at the cell surface or in an endosome or in another cell-membrane bound space.

In some embodiments, the fusogen includes a EFF-1, AFF-1, gap junction protein, e.g., a connexin (such as Cn43, GAP43, CX43) (DOI: 10.1021/jacs.6b05191), other tumor connection proteins, a homologue thereof, a fragment thereof, a variant thereof, and a protein fusion comprising one or more proteins or fragments thereof.

### Lipid Fusogens

In some embodiments, the fusogen is a fusogenic lipid, such as saturated fatty acid. In some embodiments, the saturated fatty acids have between 10-14 carbons. In some embodiments, the saturated fatty acids have longer-chain carboxylic acids. In some embodiments, the saturated fatty acids are mono-esters.

In some embodiments, the fusosome may be treated with unsaturated fatty acids. In some embodiments, the unsaturated fatty acids have between C16 and C18 unsaturated fatty acids. In some embodiments, the unsaturated fatty acids include oleic acid, glycerol mono-oleate, glycerides, diacylglycerol, modified unsaturated fatty acids, and any combination thereof.

Without wishing to be bound by theory, in some embodiments negative curvature lipids promote membrane fusion. In some embodiments, the fusosome comprises one or more negative curvature lipids, e.g., exogenous negative curvature lipids, in the membrane. In embodiments, the negative curvature lipid or a precursor thereof is added to media comprising source cells or fusosomes. In embodiments, the source cell is engineered to express or overexpress one or more lipid synthesis genes. The negative curvature lipid can be, e.g., diacylglycerol (DAG), cholesterol, phosphatidic acid (PA), phosphatidylethanolamine (PE), or fatty acid (FA).

Without wishing to be bound by theory, in some embodiments positive curvature lipids inhibit membrane fusion. In some embodiments, the fusosome comprises reduced levels of one or more positive curvature lipids, e.g., exogenous positive curvature lipids, in the membrane. In embodiments, the levels are reduced by inhibiting synthesis of the lipid, e.g., by knockout or knockdown of a lipid synthesis gene, in the source cell. The positive curvature lipid can be, e.g., lysophosphatidylcholine (LPC), phosphatidylinositol (PtdIns), lysophosphatidic acid (LPA), lysophosphatidylethanolamine (LPE), or monoacylglycerol (MAG).

### Chemical Fusogens

In some embodiments, the fusosome may be treated with fusogenic chemicals. In some embodiments, the fusogenic chemical is polyethylene glycol (PEG) or derivatives thereof.

In some embodiments, the chemical fusogen induces a local dehydration between the two membranes that leads to unfavorable molecular packing of the bilayer. In some embodiments, the chemical fusogen induces dehydration of an area near the lipid bilayer, causing displacement of aqueous molecules between cells and allowing interaction between the two membranes together.

In some embodiments, the chemical fusogen is a positive cation. Some nonlimiting examples of positive cations include Ca2+, Mg2+, Mn2+, Zn2+, La3+, Sr3+, and H+.

In some embodiments, the chemical fusogen binds to the target membrane by modifying surface polarity, which alters the hydration-dependent intermembrane repulsion.

In some embodiments, the chemical fusogen is a soluble lipid soluble. Some nonlimiting examples include oleoylglycerol, dioleoylglycerol, trioleoylglycerol, and variants and derivatives thereof.

In some embodiments, the chemical fusogen is a water-soluble chemical. Some nonlimiting examples include polyethylene glycol, dimethyl sulphoxide, and variants and derivatives thereof.

In some embodiments, the chemical fusogen is a small organic molecule. A nonlimiting example includes n-hexyl bromide.

In some embodiments, the chemical fusogen does not alter the constitution, cell viability, or the ion transport properties of the fusogen or target membrane.

In some embodiments, the chemical fusogen is a hormone or a vitamin. Some nonlimiting examples include abscisic acid, retinol (vitamin A1), a tocopherol (vitamin E), and variants and derivatives thereof.

In some embodiments, the fusosome comprises actin and an agent that stabilizes polymerized actin. Without wishing to be bound by theory, stabilized actin in a fusosome can promote fusion with a target cell. In embodiments, the agent that stabilizes polymerized actin is chosen from actin, myosin, biotin-streptavidin, ATP, neuronal Wiskott-Aldrich syndrome protein (N-WASP), or formin. See, e.g., Langmuir. 2011 Aug. 16; 27(16):10061-71 and Wen et al., Nat Commun. 2016 Aug. 31; 7. In embodiments, the fusosome comprises exogenous actin, e.g., wild-type actin or actin comprising a mutation that promotes polymerization. In embodiments, the fusosome comprises ATP or phosphocreatine, e.g., exogenous ATP or phosphocreatine.

### Small Molecule Fusogens

In some embodiments, the fusosome may be treated with fusogenic small molecules. Some nonlimiting examples include halothane, nonsteroidal anti-inflammatory drugs (NSAIDs) such as meloxicam, piroxicam, tenoxicam, and chlorpromazine.

In some embodiments, the small molecule fusogen may be present in micelle-like aggregates or free of aggregates.

### Fusosome Generation

Fusosomes Generated from Cells

Compositions of fusosomes may be generated from cells in culture, for example cultured mammalian cells, e.g., cultured human cells. The cells may be progenitor cells or non-progenitor (e.g., differentiated) cells. The cells may be primary cells or cell lines (e.g., a mammalian, e.g., human, cell line described herein). In embodiments, the cultured cells are progenitor cells, e.g., bone marrow stromal cells, marrow derived adult progenitor cells (MAPCs), endothelial progenitor cells (EPC), blast cells, intermediate progenitor cells formed in the subventricular zone, neural stem cells, muscle stem cells, satellite cells, liver stem cells, hematopoietic stem cells, bone marrow stromal cells, epidermal stem cells, embryonic stem cells, mesenchymal stem cells, umbilical cord stem cells, precursor cells, muscle precursor cells, myoblast, cardiomyoblast, neural precursor cells, glial precursor cells, neuronal precursor cells, hepatoblasts.

The cultured cells may be from epithelial, connective, muscular, or nervous tissue or cells, and combinations thereof. Fusosome can be generated from cultured cells from any eukaryotic (e.g., mammalian) organ system, for example, from the cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves)′; reproductive system (ovaries, uterus, mammary glands, testes, vas deferens, seminal vesicles, prostate); respiratory system (pharynx, larynx, trachea, bronchi, lungs, diaphragm); skeletal system (bone, cartilage), and combinations thereof. In embodiments, the cells are from a highly mitotic tissue (e.g., a highly mitotic healthy tissue, such as epithelium, embryonic tissue, bone marrow, intestinal crypts). In embodiments, the tissue sample is a highly metabolic tissue (e.g., skeletal tissue, neural tissue, cardiomyocytes).

A fusosome composition described herein may be comprised of fusosomes from one cellular or tissue source, or from a combination of sources. For example, a fusosome composition may comprise fusosomes from xenogeneic sources (e.g. animals, tissue culture of the aforementioned species' cells), allogeneic, autologous, from specific tissues resulting in different protein concentrations and distributions (liver, skeletal, neural, adipose, etc.), from cells of different metabolic states (e.g., glycolytic, respiring). A composition may also comprise fusosomes in different metabolic states, e.g. coupled or uncoupled, as described elsewhere herein.

In some embodiments, fusosomes are generated by inducing budding of a mitoparticle, pyrenocyte, exosome, liposome, lysosome, or other membrane enclosed vesicle.

In some embodiments, fusosomes are generated by inducing cell enucleation. Removing the nucleus of a cell may be performed using assays known in the art, such as genetic, chemical, mechanical methods, or combinations thereof. Enucleation refers not only to a complete removal of the nucleus but also the displacement of the nucleus from its typical location such that the cell contains the nucleus but it is non-functional.

In some embodiments, fusosomes are generated by inducing cell fragmentation. In some embodiments, cell fragmentation can be performed using the following methods, including, but not limited to: chemical methods, mechanical methods (e.g., centrifugation (e.g., ultracentrifugation, or density centrifugation), freeze-thaw, or sonication), or combinations thereof.

### Synthetic Fusosomes

Certain components of synthetic fusosomes may be generated from a cell or a tissue, for example, the fusogen, the lipid, or the cargo. In some embodiments, the fusogen may be derived from xenogeneic sources (e.g., animals, tissue culture of the aforementioned species' cells), allogeneic, autologous, from specific tissues resulting in different protein concentrations and distributions (liver, skeletal, neural, adipose, etc.), from cells of different metabolic states (e.g., glycolytic, respiring). A composition may also comprise synthetic fusosomes in different metabolic states, e.g. coupled or uncoupled, as described elsewhere herein.

Additional production techniques useful for making synthetic fusosomes, e.g., filter based vesicle production/alteration of size distribution, are described in Spuch and Navarro, Journal of Drug Delivery, vol. 2011, Article ID 469679, 12 pages, 2011. doi:10.1155/2011/469679 and Templeton et al., Nature Biotech, 15:647-652, 1997.

### Cargo

In some aspects, the disclosure provides a composition (e.g., a pharmaceutical composition) comprising (i) one or more of a chondrisome (e.g., as described in international application, PCT/US16/64251), a mitochondrion, an organelle (e.g., Mitochondria, Lysosomes, nucleus, cell membrane, cytoplasm, endoplasmic reticulum, ribosomes, vacuoles, endosomes, spliceosomes, polymerases, capsids, acrosome, autophagosome, centriole, glycosome, glyoxysome, hydrogenosome, melanosome, mitosome, myofibril, cnidocyst, peroxisome, proteasome, vesicle, stress granuole, and networks of organelles), or an enucleated cell, e.g., an enucleated cell comprising any of the foregoing, and (ii) a fusogen, e.g., a myomaker protein. In embodiments, the fusogen is present in a lipid bilayer external to the mitochondrion or chondrisome. In embodiments, the chondrisome has one or more of the properties as described, for example, in international application, PCT/US16/64251.

In some embodiments, the cargo may include one or more nucleic acid sequences, one or more polypeptides, a combination of nucleic acid sequences and/or polypeptides, one or more organelles, and any combination thereof. In some embodiments, the cargo may include one or more cellular components. In some embodiments, the cargo includes one or more cytosolic and/or nuclear components.

In some embodiments, the cargo includes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprogramming RNAs, aptamers, and any combination thereof.

In some embodiments, the cargo may include a nucleic acid. For example, RNA to enhance expression of an endogenous protein, or a siRNA that inhibits protein expression of an endogenous protein. For example, the endogenous protein may modulate structure or function in the target cells. In some embodiments, the cargo may include a nucleic acid encoding an engineered protein that modulates structure or function in the target cells. In some embodiments, the cargo is a nucleic acid that targets a transcriptional activator that modulate structure or function in the target cells.

In some embodiments, the cargo includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, DNA integration polypeptides, targeted endonucleases (e.g. Zinc-finger nucleases, transcription-activator-like nucleases (TALENs), cas9 and homologs thereof), recombinases, and any combination thereof.

In some embodiments, the cargo includes a small molecule, e.g., ions (e.g. Ca²⁺, Cl⁻, Fe²⁺), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof.

In some embodiments, the cargo includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g. Cas9-gRNA complex); multiple transcription factors, multiple epigenetic factors, reprogramming factors (e.g. Oct4, Sox2, cMyc, and Klf4); multiple regulatory RNAs; and any combination thereof.

In some embodiments, the cargo includes one or more organelles, e.g., chondrisomes, mitochondria, lysosomes, nucleus, cell membrane, cytoplasm, endoplasmic reticulum, ribosomes, vacuoles, endosomes, spliceosomes, polymerases, capsids, acrosome, autophagosome, centriole, glycosome, glyoxysome, hydrogenosome, melanosome, mitosome, myofibril, cnidocyst, peroxisome, proteasome, vesicle, stress granuole, networks of organelles, and any combination thereof.

In one aspect, the fusosome, e.g., a pharmaceutical composition of, or a composition of, comprises isolated chondrisomes (e.g., a chondrisome preparation), derived from a cellular source of mitochondria.

In another aspect, the fusosome, e.g., a pharmaceutical composition of, or a composition of, comprises isolated, modified chondrisomes (e.g., modified chondrisome preparation) derived from a cellular source of mitochondria.

In another aspect, the fusosome, e.g., a pharmaceutical composition of, or a composition of, comprises chondrisomes (e.g., chondrisome preparation) expressing an exogenous protein.

### Delivery

In certain aspects, the disclosure provides a method of delivering a membrane enclosed preparation to a target cell in a subject. In some embodiments, the method comprises administering to a subject a fusosome, e.g., a membrane enclosed preparation comprising a nucleic acid encoding a fusogen, e.g., a myomaker protein, wherein the nucleic acid is not within a cell, under conditions that allow the fusogen to be expressed on the surface of the fusosome in the subject. In some embodiments, the method further comprises administering to the subject a composition comprising an agent, e.g., a therapeutic agent, and a fusogen binding partner, optionally, comprising a carrier, e.g., a membrane, under conditions that allow fusion of the fusogen on the fusosome and the fusogen binding partner. In some embodiments, the carrier comprises a membrane, e.g., a lipid bilayer, e.g., the agent is disposed within a lipid bilayer. In some embodiments, the lipid bilayer fuses with the target cell, thereby delivering the agent to the target cell in the subject.

In some embodiments, the fusogen on a fusosome interacts with a fusogen binding partner on target membrane to induce fusion of between the fusosome and the target membrane.

In some embodiments, the fusogen interacts with a fusogen binding partner on subcellular organelles, including mitochondria.

In some embodiments, a fusogen (e.g., protein, lipid or chemical fusogen) or a fusogen binding partner is delivered to a target cell or tissue prior to, at the same time, or after the delivery of a fusosome.

In some embodiments, a fusogen (e.g., protein, lipid or chemical fusogen) or a fusogen binding partner is delivered to a non-target cell or tissue prior to, at the same time, or after the delivery of a fusosome.

In some embodiments, a nucleic acid that encodes a fusogen (e.g., protein or lipid fusogen) or a fusogen binding partner is delivered to a target cell or tissue prior to, at the same time, or after the delivery of a fusosome.

In some embodiments, a polypeptide, nucleic acid, ribonucleoprotein, or small-molecule that upregulates or downregulates expression of a fusogen (e.g., protein, lipid or chemical fusogen) or a fusogen binding partner is delivered to a target cell or tissue prior to, at the same time, or after the delivery of a fusosome.

In some embodiments, a polypeptide, nucleic acid, ribonucleoprotein, or small-molecule that upregulates or downregulates expression of a fusogen (e.g., protein, lipid or chemical fusogen) or a fusogen binding partner is delivered to a non-target cell or tissue prior to, at the same time, or after the delivery of a fusosome.

In some embodiments, the target cell or tissue is modified by (e.g. inducing stress or cell division) to increase the rate of fusion prior to, at the same time, or after the delivery of a fusosome. Some nonlimiting examples include, inducing ischemia, treatment with a chemotherapy, antibiotic, irradiation, toxin, inflammation, inflammatory molecules, anti-inflammatory molecules, acid injury, basic injury, burn, polyethylene glycol, neurotransmitters, myelotoxic drugs, growth factors, or hormones, tissue resection, starvation, and/or exercise.

In some embodiments, the target cells or tissue is treated with an epigenetic modifier, e.g., a small molecule epigenetic modifier, to increase or decrease expression of an endogenous cell surface molecule, e.g., a fusogen binding partner, e.g., an organ, tissue, or cell targeting molecule, where the cell surface molecule is a protein, glycan, lipid or low molecular weight molecule.

In some embodiments, the target cell or tissue is treated with a vasodilator (e.g. nitric oxide (NO), carbon monoxide, prostacyclin (PGI2), nitroglycerine, phentolamine) or vasoconstrictors (e.g. angiotensin (AGT), endothelin (EDN), norepinephrine)) to increase the rate of fusosome transport to the target tissue.

In some embodiments, the target cell or tissue is treated with a chemical agent, e.g., a chemotherapeutic. In such embodiments, the chemotherapeutic induces damage to the target cell or tissue that enhances fusogenic activity of target cells or tissue.

In some embodiments, the target cell or tissue is treated with a physical stress, e.g., electrofusion. In such embodiments, the physical stress destabilizes the membranes of the target cell or tissue to enhance fusogenic activity of target cells or tissue.

In some embodiments, the target cell or tissue may be treated with an agent to enhance fusion with a fusosome. For example, specific neuronal receptors may be stimulated with an anti-depressant to enhance fusogenic properties.

Compositions comprising the fusosomes described herein may be administered or targeted to the circulatory system, hepatic system, renal system, cardio-pulmonary system, central nervous system, peripheral nervous system, musculoskeletal system, lymphatic system, immune system, sensory nervous systems (sight, hearing, smell, touch, taste), digestive system, endocrine systems (including adipose tissue metabolic regulation), reproduction system.

In embodiments, a fusosome composition described herein is delivered ex-vivo to a cell or tissue, e.g., a human cell or tissue. In some embodiments, the composition is delivered to an ex vivo tissue that is in an injured state (e.g., from trauma, disease, hypoxia, ischemia or other damage).

In some embodiments, the fusosome composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). The composition improves viability, respiration, or other function of the transplant. The composition can be delivered to the tissue or organ before, during and/or after transplantation.

The fusosome compositions described herein can be used to treat a subject, e.g., a human, in need thereof. In such embodiments, the subject may be at risk, may have a symptom of, or may be diagnosed with or identified as having, a particular disease or condition (e.g., a disease or condition described herein).

In some embodiments, the source of fusosomes are from the same subject that is treated with a fusosome composition. In other embodiments, they are different. For example, the source of fusosomes and recipient tissue may be autologous (from the same subject) or heterologous (from different subjects). In either case, the donor tissue for fusosome compositions described herein may be a different tissue type than the recipient tissue. For example, the donor tissue may be muscular tissue and the recipient tissue may be connective tissue (e.g., adipose tissue). In other embodiments, the donor tissue and recipient tissue may be of the same or different type, but from different organ systems.

### Example A-1: Sonication-Mediated Generation of Fusosomes

This example describes loading of fusogens into a fusosome via sonication. Sonication methods are disclosed e.g., in Lamichhane, T N, et al., Oncogene Knockdown via Active Loading of Small RNAs into Extracellular Vesicles by Sonication. Cell Mol Bioeng, (2016), the entire contents of which are hereby incorporated by reference.

Fusosomes are prepared by any one of the methods described herein. Approximately 10⁶ fusosomes are mixed with 5-20 μg protein and incubated at room temperature for 30 minutes. The fusosome/protein mixture is then sonicated for 30 seconds at room temperature using a water bath sonicator (Brason model #1510R-DTH) operated at 40 kHz. The mixture is then placed on ice for one minute followed by a second round of sonication at 40 kHz for 30 seconds. The mixture is then centrifuged at 16,000 g for 5 minutes at 4 C to pellet the fusosomes containing protein. The supernatant containing unincorporated protein is removed and the pellet is resuspended in phosphate-buffered saline. After protein loading, the fusosomes are kept on ice before use.

### Example A-2: Generation of Fusosomes Through Protein Electroporation

This example describes electroporation of fusogens to generate fusosomes.

Approximately 5×10⁶ cells or vesicles are used for electroporation using an electroporation transfection system (Thermo Fisher Scientific). To set up a master mix, 24 μg of purified protein fusogens is added to resuspension buffer (provided in the kit). The mixture is incubated at room temperature for 10 min. Meanwhile, the cells or vesicles are transferred to a sterile test tube and centrifuged at 500×g for 5 min. The supernatant is aspirated and the pellet is resuspended in 1 ml of PBS without Ca²⁺ and Mg²⁺. The buffer with the fusogens is then used to resuspend the pellet of cells or vesicles. A cell or vesicle suspension is also used for optimization conditions, which vary in pulse voltage, pulse width and the number of pulses. After electroporation, the electroporated cells or vesicles with fusogens are washed with PBS, resuspended in PBS, and kept on ice.

See, for example, Liang et al., Rapid and highly efficiency mammalian cell engineering via Cas9 protein transfection, Journal of Biotechnology 208: 44-53, 2015.

### Example A-3: Generating and Isolating Giant Plasma Membrane Fusosomes

This example describes fusosome generation and isolation via vesiculation and centrifugation. This is one of the methods by which fusosomes may be isolated. Fusosomes are prepared as follows.

Briefly, HeLa cells that express a fusogen are washed twice in buffer (10 mM HEPES, 150 mM NaCl, 2 mM CaCl₂, pH 7.4), resuspended in a solution (1 mM DTT, 12.5 mM Paraformaldehyde, and 1 mM N-ethylmaleimide in GPMV buffer), and incubated at 37° C. for 1 h. Fusosomes are clarified from cells by first removing cells by centrifugation at 100×g for 10 minutes, and then harvesting fusosomes at 20,000×g for 1 h at 4° C. The fusosomes are resuspended in desired buffer for experimentation.

See for example, Sezgin E et al. Elucidating membrane structure and protein behavior using giant membrane plasma vesicles. Nat. Protocols. 7(6):1042-51 2012.

### Example A-4: Generating and Isolating Fusosome Ghosts

This example describes fusosome generation and isolation via hypotonic treatment and centrifugation. This is one of the methods by which fusosomes may be produced.

First, fusosomes are isolated from mesenchymal stem cells expressing fusogens (10⁹ cells) primarily by using hypotonic treatment such that the cell ruptures and fusosomes are formed. According to a specific embodiment, cells are resuspended in hypotonic solution, Tris-magnesium buffer (TM, e.g., pH 7.4 or pH 8.6 at 4° C., pH adjustment made with HCl). Cell swelling is monitored by phase-contrast microscopy. Once the cells swell and fusosomes are formed, the suspension is placed in a homogenizer. Typically, about 95% cell rupture is sufficient as measured through cell counting and standard AOPI staining. The membranes/fusosomes are then placed in sucrose (0.25 M or higher) for preservation. Alternatively, fusosomes can be formed by other approaches known in the art to lyse cells, such as mild sonication (Arkhiv anatomii, gistologii i embriologii; 1979, August, 77(8) 5-13; PMID: 496657), freeze-thaw (Nature. 1999, Dec. 2; 402(6761):551-5; PMID: 10591218), French-press (Methods in Enzymology, Volume 541, 2014, Pages 169-176; PMID: 24423265), needle-passaging (www.sigmaaldrich.com/technical-documents/protocols/biology/nuclear-protein-extraction.html) or solublization in detergent-containing solutions (www.thermofisher.com/order/catalog/product/89900).

To avoid adherence, the fusosomes are placed in plastic tubes and centrifuged. A laminated pellet is produced in which the topmost lighter gray lamina includes mostly fusosomes. However, the entire pellet is processed, to increase yields. Centrifugation (e.g., 3,000 rpm for 15 min at 4° C.) and washing (e.g., 20 volumes of Tris magnesium/TM-sucrose pH 7.4) may be repeated.

In the next step, the fusosome fraction is separated by floatation in a discontinuous sucrose density gradient. A small excess of supernatant is left remaining with the washed pellet, which now includes fusosomes, nuclei, and incompletely ruptured whole cells. An additional 60% w/w sucrose in TM, pH 8.6, is added to the suspension to give a reading of 45% sucrose on a refractometer. After this step, all solutions are TM pH 8.6. 15 ml of suspension are placed in SW-25.2 cellulose nitrate tubes and a discontinuous gradient is formed over the suspension by adding 15 ml layers, respectively, of 40% and 35% w/w sucrose, and then adding 5 ml of TM-sucrose (0.25 M). The samples are then centrifuged at 20,000 rpm for 10 min, 4° C. The nuclei sediment form a pellet, the incompletely ruptured whole cells are collected at the 40%-45% interface, and the fusosomes are collected at the 35%-40% interface. The fusosomes from multiple tubes are collected and pooled.

See for example, International patent publication, WO2011024172A2.

### Example A-5: Physical Enucleation of Fusosomes

This example describes enucleation of fusosomes via cytoskeletal inactivation and centrifugation. This is one of the methods by which fusosomes may be modified.

Fusosomes are isolated from mammalian primary or immortalized cell lines that express a fusogen. The cells are enucleated by treatment with an actin skeleton inhibitor and ultracentrifugation. Briefly, C2C12 cells are collected, pelleted, and resuspended in DMEM containing 12.5% Ficoll 400 (F2637, Sigma, St. Louis Mo.) and 500 nM Latrunculin B (ab144291, Abcam, Cambridge, Mass.) and incubated for 30 minutes at 37° C.+5% CO₂. Suspensions are carefully layered into ultracentrifuge tubes containing increasing concentrations of Ficoll 400 dissolved in DMEM (15%, 16%, 17%, 18%, 19%, 20%, 3 mL per layer) that have been equilibrated overnight at 37° C. in the presence of 5% CO₂. Ficoll gradients are spun in a Ti-70 rotor (Beckman-Coulter, Brea, Calif.) at 32,300 RPM for 60 minutes at 37 C. After ultracentrifugation, fusosomes found between 16-18% Ficoll are removed, washed with DMEM, and resuspended in DMEM.

Staining for nuclear content with Hoechst 33342 as described in Example 35 followed by the use of flow cytometry and/or imaging will be performed to confirm the ejection of the nucleus.

### Example A-6: Generating Fusosomes Through Extrusion

This example describes fusosome manufacturing by extrusion through a membrane.

Briefly, hematopoietic stem cells that express fusogens are in a 37° C. suspension at a density of 1×10⁶ cells/mL in serum-free media containing protease inhibitor cocktail (Set V, Calbiochem 539137-1 ML). The cells are aspirated with a luer lock syringe and passed once through a disposable 5 mm syringe filter into a clean tube. If the membrane fouls and becomes clogged, it is set aside and a new filter is attached. After the entire cell suspension has passed through the filter, 5 mL of serum-free media is passed through all filters used in the process to wash any remaining material through the filter(s). The solution is then combined with the extruded fusosomes in the filtrate.

Fusosomes may be further reduced in size by continued extrusion following the same method with increasingly smaller filter pore sizes, ranging from 5 mm to 0.2 mm. When the final extrusion is complete, suspensions are pelleted by centrifugation (time and speed required vary by size) and resuspended in media.

Additionally, this process can be supplemented with the use of an actin cytoskeleton inhibitor in order to decrease the influence of the existing cytoskeletal structure on extrusion. Briefly, a 1×10⁶ cell/mL suspension is incubated in serum-free media with 500 nM Latrunculin B (ab144291, Abcam, Cambridge, Mass.) and incubated for 30 minutes at 37° C. in the presence of 5% CO₂. After incubation, protease inhibitor cocktail is added and cells are aspirated into a luer lock syringe, with the extrusion carried out as previously described.

Fusosomes are pelleted and washed once in PBS to remove the cytoskeleton inhibitor before being resuspended in media.

### Example A-7: Processing Fusosomes

This example described the processing of fusosomes. Fusosomes produced via any of the described methods in the previous Examples may be further processed.

In some embodiments, fusosomes are first homogenized, e.g., by sonication. For example, the sonication protocol includes a 5 second sonication using an MSE sonicator with microprobe at an amplitude setting of 8 (Instrumentation Associates, N.Y.). In some embodiments, this short period of sonication is enough to cause the plasma membrane of the fusosomes to break up into homogenously sized fusosomes. Under these conditions, organelle membranes are not disrupted and these are removed by centrifugation (3,000 rpm, 15 min 4° C.). Fusosomes are then purified by differential centrifugation as described in Example A-5.

Extrusion of fusosomes through a commercially available polycarbonate membrane (e.g., from Sterlitech, Washington) or an asymmetric ceramic membrane (e.g., Membralox), commercially available from Pall Execia, France, is an effective method for reducing fusosome sizes to a relatively well defined size distribution. Typically, the suspension is cycled through the membrane one or more times until the desired fusosome size distribution is achieved. The fusosomes may be extruded through successively smaller pore membranes (e.g., 400 nm, 100 nm and/or 50 nm pore size) to achieve a gradual reduction in size and uniform distribution.

In some embodiments, at any step of fusosome production, though typically prior to the homogenization, sonication and/or extrusion steps, a pharmaceutical agent (such as a therapeutic), may be added to the reaction mixture such that the resultant fusosome encapsulates the pharmaceutical agent.

### Example A-8: In Vivo Delivery of Membrane Protein

This example describes fusosome fusion with a cell in vivo. In an embodiment, fusosome fusion with a cell in vivo results in delivery of an active membrane protein to the recipient cell.

In this example, the fusosomes comprise the Sendai virus HVJ-E protein as in the previous Example. In an embodiment, the fusosomes are generated to comprise the membrane protein, GLUT4. Fusosomes with and without GLUT4 are prepared as described herein.

BALB/c-nu mice are administered fusosomes comprising GLUT4, fusosomes that do not comprise GLUT4, or PBS (negative control). Mice are injected intramuscularly in the tibialis anterior muscle with fusosomes or PBS. Immediately prior to fusosome administration, mice are fasted for 12 hours and injected with [18F] 2-fluoro-2deoxy-d-glucose (18F-FDG), which is an analog of glucose that enables positron emission tomography (PET imaging). Mice are injected with 18F-FDG via the tail vein under anesthesia (2% isoflurane). PET imaging is performed using a nanoscale imaging system (1T, Mediso, Hungary). Imaging is conducted 4 hours after administration of fusosomes. Immediately after imaging, mice are sacrificed and the tibialis anterior muscle is weighed. PET images are reconstructed using a 3D imaging system in full detector mode, with all corrections on, high regularization, and eight iterations. Three-dimensional volume of interest (VOI) analysis of the reconstructed images is performed using the imaging software package (Mediso, Hungary) and applying standard uptake value (SUV) analysis. VOI fixed with a diameter of 2 mm sphere, is drawn for the tibialis anterior muscle site. The SUV of each VOI sites is calculated using the following formula: SUV=(radioactivity in volume of interest, measured as Bq/cc×body weight)/injected radioactivity.

In an embodiment, mice that are administered fusosomes comprising GLUT4 will demonstrate an increased radioactive signal in VOI as compared to mice administered PBS or fusosomes that do not comprise GLUT4.

See, also, Yang et al., Advanced Materials 29, 1605604, 2017.

### Example A-9: In Vivo Delivery of Protein

This example describes the delivery of therapeutic agents to the eye by fusosomes.

Fusosomes are produced as described herein and are loaded with a protein that is deficient in a mouse knock-out.

Fusosomes are injected subretinally into the right eye of a mouse that is deficient for the protein and vehicle control is injected into the left eye of the mice. A subset of the mice is euthanized when they reach 2 months of age.

Histology and H&E staining of the harvested retinal tissue is conducted to count the number of cells rescued in each retina of the mice (described in Sanges et al., The Journal of Clinical Investigation, 126(8): 3104-3116, 2016).

The level of the injected protein is measured in retinas harvested from mice euthanized at 2 months of age via a western blot with an antibody specific to the therapeutic protein.

In an embodiment, the left eyes of mice, which are administered fusosomes, will have an increased number of nuclei present in the outer nuclear level of the retina compared to the right eyes of mice, which are treated with vehicle. The increased protein is suggestive of complementation of the mutated protein.

### Example A-10: In Vivo Delivery of DNA

This example describes the delivery of DNA to cells in vivo via fusosomes. Delivery of DNA to cells in vivo results in the expression of proteins within the recipient cell.

Fusosome DNA delivery in vivo will demonstrates the delivery of DNA and protein expression in recipient cells within an organism (mouse).

Fusosomes that express a liver directed fusogen are prepared as described herein. Following production of the fusosome, it is additionally nucleofected with a plasmid having a sequence that codes for Cre recombinase.

Fusosomes are prepared for in vivo delivery. Fusosome suspensions are subjected to centrifugation. Pellets of the fusosomes are resuspended in sterile phosphate buffered saline for injection.

Fusosomes are verified to contain DNA using a nucleic acid detection method, e.g., PCR.

The recipient mice harbor a loxp-luciferase genomic DNA locus that is modified by CRE protein made from DNA delivered by the fusosomes to unblock the expression of luciferase (JAX #005125). The positive control for this example are offspring of recipient mice mated to a mouse strain that expresses the same protein exclusively in the liver from its own genome (albumin-CRE JAX #003574). Offspring from this mating harbor one of each allele (loxp-luciferase, albumin-CRE). Negative controls are carried out by injection of recipient mice with fusosomes not expressing fusogens or fusosomes with fusogens but not containing Cre DNA.

The fusosomes are delivered into mice by intravenous (IV) tailvein administration. Mice are placed in a commercially available mouse restrainer (Harvard Apparatus). Prior to restraint, animals are warmed by placing their cage on a circulating water bath. Once inside the restrainer, the animals are allowed to acclimate. An IV catheter consisting of a 30 G needle tip, a 3″ length of PE-10 tubing, and a 28 G needle is prepared and flushed with heparinized saline. The tail is cleaned with a 70% alcohol prep pad. Then, the catheter needle is held with forceps and slowly introduced into the lateral tail vein until blood becomes visible in the tubing. The fusosome solution (˜500K-5M fusosomes) is aspirated into a 1 cc tuberculin syringe and connected to an infusion pump. The fusosome solution is delivered at a rate of 20 uL per minute for 30 seconds to 5 minutes, depending on the dose. Upon completion of infusion, the catheter is removed, and pressure is applied to the injection site until cessation of any bleeding. Mice are returned to their cages and allowed to recover.

After fusion, the DNA will be transcribed and translated into CRE protein which will then translocates to the nucleus to carry out recombination resulting in the constitutive expression of luciferase. Intraperitoneal administration of D-luciferin (Perkin Elmer, 150 mg/kg) enables the detection of luciferase expression via the production of bioluminescence. The animal is placed into an in vivo bioluminescent imaging chamber (Perkin Elmer) which houses a cone anesthetizer (isoflurane) to prevent animal motion. Photon collection is carried out between 8-20 minutes post-injection to observe the maximum in bioluminescence due to D-luciferin pharmacokinetic clearance. A specific region of the liver is created in the software and collection exposure time set so that count rates are above 600 (in this region) to yield interpretable radiance (photons/sec/cm2/steradians) measurements. The maximum value of bioluminescent radiance is recorded as the image of bioluminescence distribution. The liver tissue is monitored specifically for radiance measurements above background (untreated animals) and those of negative controls. Measurements are carried out at 24 hours post-injection to observe luciferase activity. Mice are then euthanized and livers are harvested.

Freshly harvested tissue is subjected to fixation and embedding via immersion in 4% paraformaldehyde/0.1M sodium phosphate buffer pH7.4 at 4° C. for 1-3 hrs. Tissue is then immersed in sterile 15% sucrose/1×PBS (3 hrs. to overnight) at 4° C. Tissue is then embedded in O.C.T. (Baxter No. M7148-4). Tissue is oriented in the block appropriately for sectioning (cross-section). Tissue is then frozen in liquid nitrogen using the following method: place the bottom third of the block into the liquid nitrogen, allow to freeze until all but the center of the O.C.T. is frozen, and allow freezing to conclude on dry ice. Blocks are sectioned by cryostat into 5-7 micron sections placed on slides and refrozen for staining.

In situ hybridization is carried out (using standard methods) on tissue sections using digoxygenin labeled nucleic acid probes (for CRE DNA and luciferase mRNA detection), labeled by anti-digoxygenin fluorescent antibodies, and observed by confocal microscopy.

In embodiments, positive control animals (recombination via breeding without fusosome injection) will show bioluminescence intensity in liver as compared to untreated animals (no CRE and no fusosomes) and negative controls, while agent injected animals will show bioluminescence in liver as compared to negative controls (fusosomes without fusogen) and untreated animals.

In embodiments, detection of nucleic acid in tissue sections in agent injected animals will reveal detection of CRE recombinase and luciferase mRNA compared to negative controls and untreated animals in cells in the tissue, while positive controls will show levels of both luciferase mRNA and CRE recombinase DNA throughout the tissue.

Evidence of DNA delivery by fusosomes will be detected by in situ hybridization-based detection of the DNA and its colocalization in the recipient tissue of the animal. Activity of the protein expressed from the DNA will be detected by bioluminescent imaging. In embodiments, fusosomes will deliver DNA that will result in protein production and activity.

### Example A-11: Delivery of Mitochondria Via Protein Enhanced Fusogenic Enucleated Cells

Fusogens are imaged on a Zeiss LSM 780 inverted confocal microscope at 63× magnification 24 h following deposition in the imaging dish. Cells expressing only Mito-DsRed alone and Mito-GFP alone are imaged separately to configure acquisition settings in such a way as to ensure no signal overlap between the two channels in conditions where both Mito-DsRed and Mito-GFP are both present and acquired simultaneously. Ten regions of interest are chosen in a completely unbiased manner, with the only criteria being that a minimum of 10 cells be contained within each ROI, such that a minimum number of cells are available for downstream analysis. A given pixel in these images is determined to be positive for mitochondria if it's intensity for either channel (mito-DsRed and mito-GFP) is greater than 10% of the maximum intensity value for each respective channel across all three ROIs.

Fusion events with organelle delivery will be identified based on the criteria that >50% of the mitochondria (identified by all pixels that are either mito-GFP+ or mito-Ds-Red+) in a cell are positive for both mitoDs-Red and mito-GFP based on the above indicated threshold, which will indicate that organelles (in this case mitochondria) containing these proteins are delivered, fused and their contents intermingled. At the 24-hour time point multiple cells are expected to exhibit positive organelle delivery via fusion.

1. An apparatus for isolating one or more subcellular components comprising a cell disruption reservoir that generates at least one of a phase change, a thermal change, a physical contact force, an ultrasonic frequency, an osmotic change, a pressure change, a photothermal pulse, a magnetic field, an electromagnetic field, an electric field, and an electrical pulse through the reservoir and a separation instrument configured to specifically isolate the subcellular components based on one or more parameters selected from at least one of density, charge/pH, dielectric polarization, magnetic attraction, spectral dispersion, spectral refraction, spectral diffraction, hydrophobicity, hydrophilicity, structure (presence or absence of a structural feature), function (migration), affinity or binding, and pressure.

2. The apparatus of claim 1, wherein the cell disruption reservoir generates a photothermal pulse.

3. The apparatus of claim 1, wherein the cell disruption reservoir generates a pressure change.

4. The apparatus of claim 1, wherein the cell disruption reservoir comprises an inlet and an outlet for fluidic movement that generates the osmotic change.

5. The apparatus of claim 1, wherein the separation instrument comprises a centrifuge.

6. The apparatus of claim 1, wherein the subcellular components comprise organelles.

7. An apparatus for isolating one or more subcellular components comprising a reservoir comprising an inlet and an outlet for fluidic movement into and out of the reservoir, a pump to regulate a fluid flow through the reservoir and a separation instrument configured to specifically isolate the subcellular components based on one or more parameters selected from at least one of density, charge/pH, magnetic attraction, spectral dispersion, spectral refraction, spectral diffraction, hydrophobicity, hydrophilicity, structure (presence or absence of a structural feature), and function (migration).

8. The apparatus of claim 7, wherein the reservoir further comprises a channel having a diameter 20-90% of an input component to physically contact the input component as the pump fluidically forces the input component through the channel.

9. The apparatus of claim 7, wherein the reservoir further comprises a cell disruption homogenizing member to physically contact an input component with a physical contact force.

10. The apparatus of claim 7, wherein the separation instrument comprises a centrifuge.

11. The apparatus of claim 7, wherein the subcellular components are organelles.
